(19) United States
(12) Patent Application Publication
(10) Pub. No.: US 2024/0252678 A1
(43) Pub. Date: Aug. 1, 2024

(54) SYNTHETIC EXOSOMES (SE) FOR CNS DELIVERY OF CRISPR FOR GENE EDITING IN BRAIN DISORDERS

(71) Applicant: The Regents of the University of California, Oakland, CA (US)

(72) Inventors: Varghese John, San Francisco, CA (US); Jesus Campagna, Playa Del Rey, CA (US)

(73) Assignee: The Regents of the University of California, Oakland, CA (US)

(21) Appl. No.: 18/292,846

(22) PCT Filed: Sep. 12, 2022
(86) PCT No.: PCT/US2022/043243

§ 371 Date: Jan. 26, 2024

Related U.S. Application Data
(60) Provisional application No. 63/244,179, filed on Sep. 14, 2021.

(51) Int. Cl. A61K 48/00; A61K 9/127; A61K 38/17; C12N 9/22; C12N 9/80; C12N 15/11; C12N 15/88

(52) U.S. Cl. CPC A61K 48/0041; A61K 9/1271; A61K 38/1709; A61K 48/005; C12N 9/22; C12N 9/80; C12N 15/11; C12N 15/88; C12Y 305/04005

(57) ABSTRACT

This invention provides synthetic exosomes that contain and deliver an effective amount of a gene editor. In certain embodiments the synthetic exosome comprises a liposome formed from a lipid bilayer, where said lipid bilayer comprises: one or more phospholipids selected from the group consisting of phosphate lipids, phosphoglycerol lipids, phosphocholine lipids, and phosphoethanolamine lipids where the lipid carbon chain ranges from 3 to 24 carbon atoms: cholesterol, a cholesterol derivative, or a phytosterol; and a non-ionic surfactant; where the lipid bilayer does not contain an alcohol: the exosome is less than about 200 nm in diameter; and the exosome contains a guide RNA (gRNA) and a cytidine base editor, where the gRNA comprise a sequence that directs said n-Cas9-cytidine deaminase to edit the codon for amino acid 112 of the gene encoding ApoE4.

### CROSS-REFERENCE TO RELATED APPLICATIONS

This application claims priority to and benefit of U.S. Ser. No. 63/244,179, filed Sep. 14, 2021, which is incorporated herein by reference in its entirety for all purposes.

### STATEMENT OF GOVERNMENTAL SUPPORT

[Not Applicable]

### BACKGROUND

Apolipoprotein 84 (apoE4) has been strongly linked with Alzheimer's disease (AD) and contributes to several other neurological disorders including, but not limited to frontotemporal dementia, cerebral amyloid angiopathy (CAA) (Rannikmae et al. (2014) J. Neurol. Neurosurg. Psychiatry, 85(3): 300-305), dementia with Lewy bodies (DLB) (Tsuang et al. (2013) JAMA Neurol. 70(2): 223), tauopathy (Shi et al. (2017) Nature, 549(7673): 523-527), cerebrovascular disease (Treves et al. (1996) Alzheimer Dis. Assoc. Disord. 10(4): 189-191), multiple sclerosis (Shin et al. (2014) J. Neuroimmunol. 271(1-2): 8-17; Chapman et al. (2001) Neurol. 56(3): 312-316), and vascular dementia (Treves et al. (1996) Alzheimer Dis. Assoc. Disord. 10(4): 189-191; Liu et al. (2012) Dement. Geriatr. Cogn. Disord. 33(2-3): 96-103), as well as being related to poor neurological outcome after traumatic brain injury and hemorrhage (see, e.g., Agosta et al. (2009) Proc. Natl. Acad. Sci. USA, 106(6): 2018-2022; Bell et al. (2012) Nature, 485(7399): 512-516).

ApoE4 is an emerging target for the treatment of Alzheimer's disease and certain other pathologies (e.g., those indicated above). Various approaches include, but are not limited to reversal of hypolipidation of apoE4 (e.g. by regulating LXR/RXR to alter expression of ABCA1) (see, e.g., Safieh et al. (2019) BMC Med., 17: 64; Boehm-Cagan et al. (2014) J. Neurosci. 34(21): 7293-7301; Boehm-Cagan et al. (2016) J. Alzheimers Dis. 54(3): 1219-1233; Tachibana et al. (2016) Exp. Neurol. 277: 1-9), anti-apoE4 immunotherapy utilizing anti-ApoEr antibodies (see, e.g., Luz et al. (2016) Curr. Alzheimer Res. 13(8): 918-929), apoE4 structural correctors (see, e.g., Chen et al. (2011) J. Biol. Chem. 286(7): 5215-5221), proteases that degradeapoE4, interference with ApoE4 and downstream signaling, altering interaction of ApoE4 with Aβ and the amyloid cascade (see, e.g., Zhao et al. (2018) Biol. Psychiatry, 83(4): 347-357; Huang & Mahley (2014) Neurobiol. Dis. 72: 3-12), increasing expression of apoER2 (see, e.g., Holtzman et al. (2012) Cold Spring Harb. Perspect. Med 2(3): a006312; Chen et al. (2010) Proc. Natl. Acad. Sci. USA, 107(26): 12011-1216; Salomon-Zimri et al. (2016) J. Alzheimers Dis. 53(4): 1443-1458), use of ApoE mimetics, gene editing of ApoE4, and the like.

All of these approaches, however, are constrained by the requirement that the therapeutic moiety cross the blood brain barrier. Consequently, adequate bioavailability to cells comprising the central nervous system has contributed to limited efficacy of various approaches.

Exosomes are nano-sized vesicles (e.g., less than 200 nm) that serve as mediators for intercellular communication through the delivery of various endogenous cargos, including proteins, lipids, nucleic acids or other cellular components, to neighboring or distant cells. Exosome cargos may vary in response to different physiological or pathological conditions.

Due to the critical role of exosomes in intercellular communications in delivering cargo to recipient cells, exosomes have been investigated as a vector for the delivery of endogenous or exogenous cargo for therapeutic purposes. But the number of exosomes produced by cells is limited, which hampers their application and use as transport system for therapeutic moieties. Additionally, the production of exosomes from cells is a tedious, low yield process that is often not well-controlled. Moreover, the essential components of active exosomes are not well established. Finally, fundamental mechanisms of exosomal delivery are currently unclear. Such issues have challenged the development of exosomes for the delivery of therapeutic agents.

### SUMMARY

One approach to the treatment and/or prophylaxis of Alzheimer's disease and other pathologies associated with ApoE4, is to decrease the risk conferred by the E4 gene by converting it to ApoE3 by use of CRISPR base editing technology, which allows editing in both dividing and non-dividing cells, critical for a significant effect in the brain. For gene editing to be possible in AD, CRISPR components must be delivered across the blood-brain barrier (BBB). We have developed a brain delivery technology involving microfluidic-reactor-based synthesis of deformable nanovesicles the size of natural exosomes (<150 nm), which we call “synthetic exosomes” (SEs) that encapsulate macromolecules. The SEs described herein can be used to deliver a gene editor and associated guide RNA across the blood brain barrier and into neural cells to effect editing of a gene encoding ApoE4.

Accordingly, various embodiments provided herein may include, but need not be limited to, one or more of the following:

Embodiment 1: A synthetic exosome capable of delivering a therapeutic moiety across the blood brain barrier into the central nervous system (CNS), said synthetic exosome comprising:
a liposome formed from a lipid bilayer, where said lipid bilayer comprises:
   one or more phospholipids selected from the group consisting of phosphate lipids, phosphoglycerol lipids, phosphocholine lipids, and phosphoethanolamine lipids where the lipid carbon chain ranges from 3 to 24 carbon atoms;
   cholesterol, a cholesterol derivative, or a phytosterol; and
   a non-ionic surfactant;
wherein:
   said lipid bilayer does not contain an alcohol;
   said exosome is less than about 200 nm in diameter; and
wherein said synthetic exosome contains a guide RNA (gRNA) and a cytidine base editor, where said gRNA comprise a sequence that directs said n-Cas9-cytidine deaminase to edit the codon for amino acid 112 of the gene encoding ApoE4, and/or said synthetic exosome contains one or more proteins selected from the group consisting of SE-sAPPα, iduronidase (IDUA), SE(ζ−)-IDUA, SE(ζ+)-IDUA, acid sphingomylienase (ASM), and SE-ASM.

Embodiment 2: The synthetic exosome of embodiment 1, wherein said synthetic exosome contains a guide RNA (gRNA) and a cytidine base editor, where said gRNA comprise a sequence that directs said n-Cas9-cytidine deaminase to edit the codon for amino acid 112 of the gene encoding ApoE4.

Embodiment 3: The synthetic exosome of embodiment 2, wherein said cytidine base editor comprises comprising an nCas9-cytidine deaminase.

Embodiment 4: The synthetic exosome of embodiment 3, wherein said cytidine base editor selected from the group consisting of BE3, BE4, BE4max, and AncBE4max.

Embodiment 5: The synthetic exosome of embodiment 4, wherein said cytidine base editor comprises BE3.

Embodiment 6: The synthetic exosome of embodiment 4, wherein said cytidine base editor comprises BE4.

Embodiment 7: The synthetic exosome of embodiment 4, wherein said cytidine base editor comprises BE4max.

Embodiment 8: The synthetic exosome of embodiment 4, wherein said cytidine base editor comprises AncBE4max.

Embodiment 9: The synthetic exosome according to any one of embodiments 3-8. wherein said guide RNA is complexed to said nCas9-cytidine deaminase.

Embodiment 10: The synthetic exosome according to any one of embodiments 1-9, wherein the gRNA sequence comprises the same 20 nt sequence as the protospacer sequence abutting the PAM as shown in Table 1.

Embodiment 11: The synthetic exosome according to any one of embodiments 1-9, wherein the gRNA sequence comprises the same sequence as the protospacer sequence abutting the PAM as shown in Table 2.

Embodiment 12: The synthetic exosome according to any one of embodiments 1-9, wherein the gRNA sequence comprises or consists of the crRNA sequence GGACGUGCGCGGCCGCCUGG (SEQ ID NO:17).

Embodiment 13: The synthetic exosome according to any one of embodiments 10-12, wherein said gRNA further comprises a tracrRNA that specifically binds Cas9 protein.

Embodiment 14: The synthetic exosome of embodiment 13, wherein said tracrRNA comprises an 80 nt sequence.

Embodiment 15: The synthetic exosome of embodiment 13, wherein said tracrRNA sequence comprises or consists of the sequence AGCAUAGCAAGUUUAAAUAAGGCUAGUC CGUUAUCAACUUGAAAAAGUGGCACCGAGUCGGUGCUUU (SEQ ID NO:18).

Embodiment 16: The synthetic exosome according to any one of embodiments 12-15, wherein said gRNA sequence comprises a linker RNA joining said tracrRNA sequence to said crRNA sequence.

Embodiment 17: The synthetic exosome of embodiment 16, wherein said linker RNA ranges in length from about 6 nt or from about 8 nt up to about 20 nt, or up to about 16 nt, or up to about 12 nt, or up to about 10 nt.

Embodiment 18: The synthetic exosome of embodiment 17, wherein said linker comprises a sequence complementary to a region of said tracrRNA.

Embodiment 19: The synthetic exosome according to any one of embodiments 1-16, wherein the ratio of Cas9 to gRNA in said exosome ranges from about 0.5 to 2 to about 2 to 0.5.

Embodiment 20: The synthetic exosome of embodiment 19, wherein the ratio of Cas9 to gRNA in said exosome ranges from about 1:1 to about 2:1.

Embodiment 21: The synthetic exosome of embodiment 20, wherein the ratio of Cas9 to gRNA in said exosome is about 1.5:1.

Embodiment 22: The synthetic exosome of embodiment 1, wherein said exosome is about 50 nm up to about 200 nm in diameter, or about 50 nm up to about 150 nm in diameter, or about 50 nm up to about 110 nm.

Embodiment 23: The synthetic exosome according to any one of embodiments 1-21, wherein said exosome is less than about 150 nm in diameter.

Embodiment 24: The synthetic exosome according to any one of embodiment 1-23, wherein said synthetic exosome is capable of crossing the blood brain barrier without substantially leaking said nCas9-cytidine deaminase and/or said gRNA.

Embodiment 25: The synthetic exosome according to any one of embodiments 1-24, wherein said lipid bilayer consists of said one or more phospholipids, said cholesterol or cholesterol derivative or a phytosterol; and said non-ionic surfactant.

Embodiment 26: The synthetic exosome according to any one of embodiments 1-25, wherein said exosome is capable of crossing the blood/brain barrier (BBB) and delivering the nCas9-cytidine deaminase and gRNA into a brain cell in sufficient amount to edit the genome of said brain cell.

Embodiment 27: The synthetic exosome of embodiment 26, wherein said exosome is capable of crossing the blood/brain barrier (BBB) and delivering the nCas9-cytidine deaminase and gRNA contained therein to the central nervous system without losing more than about 40%, or without losing more than 30%, or without losing more than 20%, or without losing more than 10%, or without losing more than 5%, or without losing more than 3%, or without losing more than 1% of the nCas9-cytidine deaminase and gRNA.

Embodiment 28: The synthetic exosome of embodiment 1, wherein said synthetic exosome contains one or more proteins selected from the group consisting of SE-sAPPα, iduronidase (IDUA), SE(ζ−)-IDUA, SE(ζ+)-IDUA, acid sphingomylienase (ASM), and SE-ASM.

Embodiment 29: The synthetic exosome of embodiment 28, wherein said synthetic exosome contains one or more proteins selected from the group consisting of SE-SAPPa.

Embodiment 30: The synthetic exosome according to any one of embodiments of embodiment 28-29, wherein said synthetic exosome contains iduronidase (IDUA).

Embodiment 31: The synthetic exosome according to any one of embodiments of embodiment 28-30, wherein said synthetic exosome contains SE(ζ−)-IDUA.

Embodiment 32: The synthetic exosome according to any one of embodiments of embodiment 28-31, wherein said synthetic exosome contains SE(ζ+)-IDUA.

Embodiment 33: The synthetic exosome according to any one of embodiments of embodiment 28-32, wherein said synthetic exosome contains acid sphingomylienase (ASM).

Embodiment 34: The synthetic exosome according to any one of embodiments of embodiment 28-33, wherein said synthetic exosome contains SE-ASM.

Embodiment 35: The synthetic exosome according to any one of embodiments 1-41, wherein said lipid bilayer does not contain an alcohol.

Embodiment 36: The synthetic exosome of embodiment 35, wherein said lipid bilayer does not contain ethanol.

Embodiment 37: The synthetic exosome according to any one of embodiments 1-36, wherein said bilayer does not contain glutathione-maleimide-PEG2000-distearoyl phosphatidyl ethanolamine.

Embodiment 38: The synthetic exosome according to any one of embodiments 1-37, wherein said exosome is not a transferosome.

Embodiment 39: The synthetic exosome according to any one of embodiments 1-38, wherein said exosome is not an ethosome.

Embodiment 40: The synthetic exosome according to any one of embodiments 1-39, wherein the molar ratio of total phospholipid to cholesterol, cholesterol, or phytosterol ranges from about 6-10 moles of total phospholipid to about 1-3 moles of cholesterol.

Embodiment 41: The synthetic exosome according to any one of embodiments 1-40, wherein the amount of surfactant ranges from about 1%, or from about 3%, or from about 5%, or from about 8% up to about 18%, or up to about 15%, or up to about 13%, or up to about 10% (wt/wt).

Embodiment 42: The synthetic exosome according to any one of embodiments 1-41, wherein said surfactant comprise one or more surfactants selected from the group consisting of Span 80, Tween 20, BRIJ® 76 (stearyl poly(10)oxy ethylene ether), BRIJ® 78 (stearyl poly(20)oxyethylene ether), BRIJ® 96 (oleyl poly(10)oxy ethylene ether), and BRIJ® 721 (stearyl poly (21) oxyethylene ether).

Embodiment 43: The synthetic exosome of embodiment 42, wherein said surfactant comprises or consists of Span 80.

Embodiment 44: The synthetic exosome of embodiment 43, wherein the lipid bilayer comprises about 10% to about 20%, or about 15% Span 80 by weight.

Embodiment 45: The synthetic exosome according to any one of embodiments 1-44, wherein said cholesterol, cholesterol derivative, or phytosterol comprises or consists of cholesterol.

Embodiment 46: The synthetic exosome according to any one of embodiments 1-44, wherein said cholesterol, cholesterol derivative, or phytosterol comprises or consists of a cholesterol derivative selected from the group consisting of cholesterol hemisuccinate, lysine-based cholesterol (CHLYS), 20-hydroxychloesterol, 22-hydroxycholesterol, 24-hydroxycholesterol, 25-hydroxy cholesterol, 27-hydroxycholesterol, cholesteryl succinate, cholic succinate, cholic tri-succinate, lithocholic succinate, chenodesoxycholic bis-scuccinate, and Hederoside.

Embodiment 47: The synthetic exosome according to any one of embodiments 1-44, wherein said cholesterol, cholesterol derivative comprises or consists cholesterol hemisuccinate.

Embodiment 48: The synthetic exosome according to any one of embodiments 1-44, wherein said cholesterol, cholesterol derivative, or phytosterol comprises or consists of a phytosterol.

Embodiment 49: The synthetic exosome of embodiment 48, wherein said phytosterol comprises a 9,10-secosteroid.

Embodiment 50: The synthetic exosome of embodiment 49, wherein said 9,10 secosteroid comprises a compound selected from the group consisting of vitamin D3, vitamin D2, calcipotriol.

Embodiment 51: The synthetic exosome of embodiment 48, wherein said phytosterol comprises a C-24 alkyl steroid.

Embodiment 52: The synthetic exosome of embodiment 51, wherein said C-24 alkyl steroid comprises a compound selected from the group consisting of stigmasterol, and β-sitosterol.

Embodiment 53: The synthetic exosome of embodiment 48, wherein said phytosterol comprises a pentacyclic steroid.

Embodiment 54: The synthetic exosome of embodiment 53, wherein said pentacyclic steroid comprises a compound selected from the group consisting of betulin, lupeol, ursolic acid, and oleanolic acid.

Embodiment 55: The synthetic exosome according to any one of embodiments 1-54, wherein said cholesterol, cholesterol derivative, or phytosterol is pegylated.

Embodiment 56: The synthetic exosome according to any one of embodiments 1-55, wherein said one or more phospholipids comprises one or more phospholipids selected from the group consisting of dihexanoyl-sn-glycero-3-phosphate (DHPA), didecanoyl-sn-glycero-3-phosphate (DDPA), distearoyl-sn-glycero-3-phosphate (DTPA), and dihexadecyl phosphate (DHP).

Embodiment 57: The synthetic exosome according to any one of embodiments 1-56, wherein said one or more phospholipids comprises one or more phosphoglycerol lipids selected from the group consisting of dihexanoyl-sn-glycero-3-phospho-(1′-rac-glycerol) (DHPG), dilauroyl-sn-glycero-3-phospho-(1′-rac-glycerol) (DLPG), and distearoyl-sn-glycero-3-phospho-(1′-rac-glycerol) (DTPG).

Embodiment 58: The synthetic exosome according to any one of embodiments 1-57, wherein said one or more phospholipids comprises one or more phosphocholine lipids selected from the group consisting of dipropionyl-sn-glycero-3-phosphocholine (PC), diheptanoyl-sn-glycero-3-phosphocholine (DHPC), dimyristoyl-sn-glycero-3-phosphocholine (DMPC), and dilignoceroyl-sn-glycero-3-phosphocholine (DGPC).

Embodiment 59: The synthetic exosome according to any one of embodiments 1-57, wherein said one or more phospholipids comprises one or more phosphoethanolamine lipids selected from the group consisting of sihexanoyl-sn-glycero-3-phosphoethanolamine (DHPE), and distearoyl-sn-glycero-3-phosphoethanolamine (DTPE).

Embodiment 60: The synthetic exosome according to any one of embodiments 1-57, wherein said one or more phospholipids comprises one or more phosphoethanolamine-PEG lipids selected from the group consisting of dipalmitoyl-sn-glycero-3-phospho(ethylene glycol) (DPPEG1), dimyristoyl-sn-glycero-3-phosphoethanolamine-N-[methoxy(polyethylene glycol)-350 (DMPEG350), distearoyl-sn-glycero-3-phosphoethanolamine-N-[methoxy(polyethylene glycol)-350] (DTPEG350), dimyristoyl-sn-glycero-3-phosphoethanolamine-N-[methoxy(polyethylene glycol)-550] (DMPEG550), and dimyristoyl-sn-glycero-3-phosphoethanolamine-N-[methoxy(polyethylene glycol)-1000] (DMPEG1000).

Embodiment 61: The synthetic exosome according to any one of embodiments 1-57, wherein said one or more phospholipids comprises one or more phospholipids selected from the group consisting of dioleoyl-sn-glycero-3-phosphocholine (N-aminoethyl) (PC-NH₂), diphytanoyl-sn-glycero-3-phosphoethanolamine, dioleoyl-3-trimethylammonium-propane (DOTAP), distearoyl-3-trimethylammonium-propane (DSTAP), dimyristoyl-3-trimethylammonium-propane (DMTAP), and di-O-octadecyl-sn-glycero-3-phosphocholin (DOPC).

Embodiment 62: The synthetic exosome according to any one of embodiments 1-61, wherein said one or more phospholipids is functionalized with a targeting moiety selected from the group consisting of transferrin, an amino acid, a blood brain barrier targeting antibody, insulin, folic acid, and low density lipoprotein receptor related protein 1.

Embodiment 63: The synthetic exosome according to any one of embodiments 1-62, wherein said lipid bilayer comprises or consists of:
said surfactant; and
3:2:1 molar ratio (DHPA:DHP:CH), 1:5:1 molar ratio (DHPG:DHPA:CH), 2:5:1:2 molar ratio (DHPG:DHPA:PC-NH2:CH), or 2:4:1:1:2 molar ratio (DHPG:DHPA:PC-NH2:DMPEG350:CH) to provide synthetic exosomes having a zeta potential of about −20 mV or lower.

Embodiment 64: The synthetic exosome according to any one of embodiments 1-62, wherein said lipid bilayer comprises or consists of:
said surfactant; and
2:2:1 molar ratio (DHPG:DHPC:CH), 4:4:1:2 molar ratio (DHPG:DHPA:PC-NH2:CH), 2:2:1 molar ratio (DHPG:DHPA:CH), 4:4:1:1:2 molar ratio (DHPG:DHPA:PC-NH2:DMPEG550:CH), 2:2:1 molar ratio (DHPG:DTPE:CH), 2:2:1 molar ratio (DHPG:DMTAP:CH), 4:4:1:2 molar ratio (DHPG:DMTAP:PC-NH2:CH), or 4:4:1:1:2 molar ratio (DHPG:DMTAP:PC-NH2:DMPEG550:CH) to provide synthetic exosomes having a zeta potential ranging from about −20 mV to about 20 mV.

Embodiment 65: The synthetic exosome according to any one of embodiments 1-62, wherein said lipid bilayer comprises or consists of:
said surfactant; and 2:4:1 molar ratio (DHPC:DTPE:CH), 2:4:1 molar ratio (DHPC:DOTAP:CH), 2:4:1:2 molar ratio (DHPC:DMTAP:PC-NH2:CH), or 2:4:1:1:2 molar ratio (DHPC:DMTAP:PC-NH2:DMPEG350:CH) to provide synthetic exosomes having a zeta potential of about 20 mV or greater.

Embodiment 66: The synthetic exosome according to any one of embodiments 1-62, wherein said lipid bilayer comprises or consists of:
said surfactant; and 4:2:1 molar ratio (DTPA:DHP:CH), 1:5:1 molar ratio (DTPG:DTPA:CH), 1:5:1:2 molar ratio (DTPG:DTPA:PC-NH2:CH), or 1:4:1:1:2 molar ratio (DTPG:DTPA:PC-NH2:DMPEG350:CH) to provide synthetic exosomes having a zeta potential of about −20 mV or lower.

Embodiment 67: The synthetic exosome according to any one of embodiments 1-62, wherein said lipid bilayer comprises or consists of:
said surfactant; and 2:2:1 molar ratio (DTPG:DGPC:CH), 4:4:1:2 molar ratio (DTPG:DDPA:PC-NH2:CH), 2:2:1 molar ratio (DTPG:DDPA:CH), 4:4:1:1:2 molar ratio (DTPG:DDPA:PC-NH2:DMPEG550:CH), 2:2:1 molar ratio (DTPG:DTPE:CH), 2:2:1 molar ratio (DTPG:DMTAP:CH), 4:4:1:2 molar ratio (DTPG:DMTAP:PC-NH2:CH), or 4:4:1:1:2 molar ratio (DTPG:DMTAP:PC-NH2:DMPEG550:CH) to provide synthetic exosomes having a zeta potential ranging from about −20 mV to about 20 mV.

Embodiment 68: The synthetic exosome according to any one of embodiments 1-62, wherein said lipid bilayer comprises or consists of:
said surfactant; and 2:4:1 molar ratio (DMPC:DTPE:CH), 2:4:1 molar ratio (DMPC:DOTAP:CH), 2:4:1:2 molar ratio (DMPC:DMTAP:PC-NH2:CH), or 2:4:1:1:2 molar ratio (DMPC:DMTAP:PC-NH2:DMPEG350:CH) to provide synthetic exosomes having a zeta potential of about 20 mV or greater.

Embodiment 69: The synthetic exosome according to any one of embodiments 63-68, wherein CH is cholesterol.

Embodiment 70: The synthetic exosome according to any one of embodiments 63-68, wherein CH is a cholesterol derivative.

Embodiment 71: The synthetic exosome of embodiment 70, wherein said cholesterol derivative is selected from the group consisting of cholesterol hemisuccinate, lysine-based cholesterol (CHLYS), 20-hydroxychloesterol, 22-hydroxycholesterol, 24-hydroxycholesterol, 25-hydroxy cholesterol, 27-hydroxycholesterol, cholesteryl succinate, cholic succinate, cholic tri-succinate, lithocholic succinate, chenodesoxycholic bis-scuccinate, and Hederoside.

Embodiment 72: The synthetic exosome of embodiment 71, wherein CH is cholesterol hemisuccinate.

Embodiment 73: The synthetic exosome according to any one of embodiments 63-68, wherein CH is a phytosterol.

Embodiment 74: The synthetic exosome of embodiment 73, wherein CH is a C-24 alkyl steroid.

Embodiment 75: The synthetic exosome of embodiment 73, wherein CH is a C-24 alkyl steroid.

Embodiment 76: The synthetic exosome according to any one of embodiments 1-75, wherein a targeting moiety comprising or consisting of transferrin or transferrin receptor binding peptides, or folic acid, or an amino acid, or insulin, or a low density lipoprotein receptor related protein 1 is attached to said exosome.

Embodiment 77: The synthetic exosome according to any one of embodiments 1-75, wherein a targeting moiety comprising or consisting of a blood brain barrier targeting antibody is attached to said exosome.

Embodiment 78: The synthetic exosome according to any one of embodiments 1-75, wherein said exosome is attached to an antibody or a ligand that binds to a moiety selected from the group consisting of a transferrin receptor, an insulin receptor, an insulin growth factor receptor (IGFIR), a low-density lipoprotein (LDL) receptor, basigin, Glut1, CD98hc, and TMEM30A(cdc50A).

Embodiment 79: The synthetic exosome according to any one of embodiments 1-75, wherein said exosome is attached to an antibody or a ligand that binds to a cell surface marker.

Embodiment 80: The synthetic exosome of embodiment 79, wherein said cell surface marker is a marker of neural or glial cells.

Embodiment 81: The synthetic exosome of embodiment 79, wherein said cell surface marker is selected from the group consisting of CD63, CD81, CD9, and CD171, and is incorporated in the lipid bilayer of said exosome.

Embodiment 82: The synthetic exosome according to any one of embodiments 1-81, wherein a said synthetic exosome is pegylated.

Embodiment 83: A pharmaceutical formulation comprising:
a synthetic exosome according to any one of embodiments 1-82; and
a pharmaceutically acceptable carrier.

Embodiment 84: The formulation of embodiment 83, wherein said formulation is compounded for delivery by route selected from the group consisting of oral delivery, isophoretic delivery, subdermal delivery, transdermal delivery, parenteral delivery, aerosol administration, administration via inhalation, intravenous administration, and rectal administration.

Embodiment 85: The formulation of embodiment 84, wherein said formulation is compounded for oral administration.

Embodiment 86: The formulation of embodiment 84, wherein said formulation is compounded for transdermal administration.

Embodiment 87: The formulation of embodiment 86, wherein said formulation is provided as a transdermal patch.

Embodiment 88: The formulation of embodiment 84, wherein said formulation is compounded for systemic administration.

Embodiment 89: The formulation according to any one of embodiments 83-88, wherein said formulation is a unit dosage formulation.

Embodiment 90: A kit comprising a container containing a synthetic exosome according to any one of embodiments 1-82, and/or a pharmaceutical formulation according to any one of embodiments 83-89.

Embodiment 91: The kit of embodiment 90, wherein said kit comprises instructional materials teaching the use of said synthetic exosome to mitigate one or more symptoms associated with a disease characterized by the presence of and/or elevated presence of Apoe4, and/or the use of said composition in delaying or preventing the onset of one or more of said symptoms of said disease.

Embodiment 92: A method of reducing the risk, lessening the severity, or delaying the progression or onset of a pathology characterized by the presence of and/or elevated presence of Apoe4 in the brain of a mammal, said method comprising:
administering, or causing to be administered, to said mammal synthetic exosome according to any one of embodiments 1-82, and/or a pharmaceutical formulation according to any one of embodiments 83-89 in an amount sufficient to reduce the risk, lessen the severity, or delay the progression or onset of said disease.

Embodiment 93: The method of embodiment 92, wherein said pathology comprises a pathology selected from the group consisting of Alzheimer's disease (AD), frontotemporal dementia, cerebral amyloid angiopathy (CAA), dementia with Lewy bodies (DLB), tauopathy, cerebrovascular disease, multiple sclerosis, vascular dementia, and traumatic brain injury hemorrhage, Parkinson's disease, Age related macular degeneration (AMD), dementia after stroke, cardiovascular disease, and/or LDL levels.

Embodiment 94: A method of preventing or delaying the onset of a pre-Alzheimer's condition and/or cognitive dysfunction, and/or ameliorating one or more symptoms of a pre-Alzheimer's condition and/or cognitive dysfunction, or preventing or delaying the progression of a pre-Alzheimer's condition or cognitive dysfunction to Alzheimer's disease in a mammal, said method comprising:
administering, or causing to be administered, to said mammal an effective amount of a synthetic exosome according to any one of embodiments 1-82, and/or a pharmaceutical formulation according to any one of embodiments 83-89.

Embodiment 95: A method of treating a pathology in a mammal selected from the group consisting of selected from the group consisting of Alzheimer's disease (AD), frontotemporal dementia, cerebral amyloid angiopathy (CAA), dementia with Lewy bodies (DLB), tauopathy, cerebrovascular disease, multiple sclerosis, vascular dementia, and traumatic brain injury and/or hemorrhage, said method comprising:
administering, or causing to be administered, to said mammal an effective amount of a synthetic exosome according to any one of embodiments 1-82, and/or a pharmaceutical formulation according to any one of embodiments 83-89.

Embodiment 96: The method according to any one of embodiments 92-95, wherein said pathology is Alzheimer's disease.

Embodiment 97: The method of embodiment 96, wherein said mammal is a mammal at risk for Alzheimer's disease.

Embodiment 98: The method of embodiment 97, wherein said mammal has a family history of Alzheimer's disease.

Embodiment 99: The method of embodiment 98, wherein said mammal has a genetic risk factor for Alzheimer's disease.

Embodiment 100: The method of embodiment 99, wherein the mammal has a familial Alzheimer's disease (FAD) mutation.

Embodiment 101: The method of embodiment 99, wherein said mammal has one copy of the ApoE4 allele.

Embodiment 102: The method of embodiment 99, wherein said mammal has two copies of the ApoE4 allele.

Embodiment 103: The method according to any one of embodiments 92-102, wherein said mammal is a human.

Embodiment 104: The method according to any one of embodiments 92-103, wherein said administering or causing to be administered comprises administering said synthetic exosome or said pharmaceutical formulation to said mammal.

Embodiment 105: The method according to any one of embodiments 92-103, wherein said administering or causing to be administered comprises prescribing or providing synthetic exosome or said pharmaceutical formulation to said mammal.

### Definitions

The term “about” when used with respect to a numerical value refers to that value ±10%, or ±5%, ±3%, or ±2%, or ±1% of that value. In certain embodiments about refers to ±10% of the value. In certain embodiments about refers to ±5% of the value. In certain embodiments about refers to ±2% of the value.

The terms “subject,” “individual,” and “patient” may be used interchangeably and typically a mammal, in certain embodiments a human or a non-human primate. While the compositions and methods are described herein with respect to use in humans, they are also suitable for animal, e.g., veterinary use. Thus, certain illustrative organisms include, but are not limited to humans, non-human primates, canines, equines, felines, porcines, ungulates, lagomorphs, and the like. Accordingly, certain embodiments contemplate the compositions and methods described herein for use with domesticated mammals (e.g., canine, feline, equine), laboratory mammals (e.g., mouse, rat, rabbit, hamster, guinea pig), and agricultural mammals (e.g., equine, bovine, porcine, ovine), and the like. The term “subject” does not require one to have any particular status with respect to a hospital, clinic, or research facility (e.g., as an admitted patient, a study participant, or the like). Accordingly, in various embodiments, the subject can be a human (e.g., adult male, adult female, adolescent male, adolescent female, male child, female child) under the care of a physician or other health worker in a hospital, psychiatric care facility, as an outpatient, or other, clinical context. In certain embodiments, the subject may not be under the care or prescription of a physician, or other, health worker. In certain embodiments the subject may not be under the care a physician or health worker and, in certain embodiments, may self-prescribe and/or self-administer the compounds described herein.

As used herein, the phrase “a subject in need thereof” refers to a subject that suffers or is at a risk of suffering (e.g., pre-disposed such as genetically pre-disposed) from the diseases or conditions listed herein (e.g., Alzheimer's disease or other neurological pathologies).

A “prophylactically effective amount” refers to an amount effective, at dosages and for periods of time necessary, to achieve the desired prophylactic result. Typically, but not necessarily, since a prophylactic dose is used in subjects prior to or at an earlier stage of disease. In certain embodiments the prophylactically effective amount may be less than the therapeutically effective amount.

The terms “treatment,” “treating,” or “treat” as used herein, refer to actions that produce a desirable effect on the symptoms or pathology of a disease or condition, particularly those that can be effected utilizing the multi-component formulation(s) described herein, and may include, but are not limited to, even minimal changes or improvements in one or more measurable markers of the disease or condition being treated. Treatments also refers to delaying the onset of, retarding or reversing the progress of, reducing the severity of, or alleviating or preventing either the disease or condition to which the term applies, or one or more symptoms of such disease or condition. “Treatment,” “treating,” or “treat” does not necessarily indicate complete eradication or cure of the disease or condition, or associated symptoms thereof. In one embodiment, treatment comprises improvement of at least one symptom of a disease being treated. The improvement may be partial or complete. The subject receiving this treatment is any subject in need thereof. Exemplary markers of clinical improvement will be apparent to persons skilled in the art.

An “effective amount” refers to an amount effective, at dosages and for periods of time necessary, to achieve the desired therapeutic or prophylactic result. A “therapeutically effective amount” of an SE containing a gene editor that targets ApoE4 may vary according to factors such as the disease state, age, sex, and weight of the individual, and the ability of the treatment to elicit a desired response in the individual. A therapeutically effective amount is also one in which any toxic or detrimental effects of a treatment are substantially absent or are outweighed by the therapeutically beneficial effects. The term “therapeutically effective amount” refers to an amount of one or more active agents described herein (e.g., a synthetic exosome (SE) a gene editor that targets ApoE4) or composition comprising the same that is effective to “treat” a disease or disorder in a mammal (e.g., a patient). In one embodiment, a therapeutically effective amount is an amount sufficient to improve at least one symptom associated with a neurological disorder, improve neurological function, improve cognition, or one or more markers of a neurological disease, or to enhance the efficacy of one or more pharmaceuticals administered for the treatment or prophylaxis of a neurodegenerative pathology. In certain embodiments, an effective amount is an amount sufficient alone, or in combination with a pharmaceutical agent to prevent advancement or the disease, delay progression, or to cause regression of a disease, or which is capable of reducing symptoms caused by the disease.

The term “mitigating” refers to reduction or elimination of one or more symptoms of that pathology or disease, and/or a reduction in the rate or delay of onset or severity of one or more symptoms of that pathology or disease, and/or the prevention of that pathology or disease.

As used herein, the phrases “improve at least one symptom” or “improve one or more symptoms” or equivalents thereof, refer to the reduction, elimination, or prevention of one or more symptoms of pathology or disease. Illustrative symptoms of pathologies treated, ameliorated, or prevented by the compositions (active agents) described herein (e.g., a SE containing a gene editor that targets ApoE4) include, but are not limited to, reduction, elimination, or prevention of one or more markers that are characteristic of the pathology or disease (e.g., of total-Tau (tTau), phospho-Tau (pTau), APPneo, soluble Aβ40, pTau/Aβ42 ratio and tTau/Aβ42 ratio, and/or an increase in the CSF of levels of one or more components selected from the group consisting of Aβ42/Aβ40 ratio, Aβ42/Aβ38 ratio, sAPPα, βAPPα/βAPP ratio, βAPPα/Aβ40 ratio, βAPPα/Aβ42 ratio, etc.) and/or reduction, stabilization or reversal of one or more diagnostic criteria (e.g., clinical dementia rating (CDR)). Illustrative measures for improved neurological function include but are not limited to the use of the mini-mental state examination (MMSE) or Folstein test (a questionnaire test that is used to screen for cognitive impairment), the General Practitioner Assessment of Cognition (GPCOG), a brief screening test for cognitive impairment described by Brodaty et al., (2002) Geriatrics Society 50(3): 530-534, and the like.

As used herein, “administer” or “administering” means to introduce, such as to introduce to a subject a compound or composition. The term is not limited to any specific mode of delivery, and can include, for example, subcutaneous delivery, intravenous delivery, intramuscular delivery, intracisternal delivery, delivery by infusion techniques, transdermal delivery, oral delivery, nasal delivery, and rectal delivery. Furthermore, depending on the mode of delivery, the administering can be carried out by various individuals, including, for example, a health-care professional (e.g., physician, nurse, etc.), a pharmacist, or the subject (i.e., self-administration).

The phrase “cause to be administered” refers to the actions taken by a medical professional (e.g., a physician), or a person prescribing and/or controlling medical care of a subject, that control and/or determine, and/or permit the administration of the agent(s)/compound(s) at issue to the subject. Causing to be administered can involve diagnosis and/or determination of an appropriate therapeutic or prophylactic regimen, and/or prescribing particular agent(s)/compounds (e.g., synthetic exosomes comprising a gene editor that targets ApoE4) for a subject. Such prescribing can include, for example, drafting a prescription form, annotating a medical record, and the like.

A “Cas9 nickase” or “nCas9” or “Cas9n” refers to a CRISPR Cas9 nuclease in which one of two Cas9 nuclease domains is mutated to produce a CRISPR Cas9 nickase. Nickases create a single-strand rather than a double-strand break.

### BRIEF DESCRIPTION OF THE DRAWINGS

FIG. 1, panels A-F, schematically illustrates one embodiment of a base editing scheme. Panel A) Cas9-nickase (NCas9) with linked cytosine deaminase (APOBEC1) and UGI is guided by the gRNA to the E4 region and the target (panel C). As shown in panels B and C, the cytosine is deaminated and endogenous correction of the U by uracil-n-glycosylate is inhibited by the linked UG inhibitor (UGI). A single stranded (ss) nick is cut by nCas9 and the U is converted to T by DNA repair. Panel D) This gRNA directed alignment with the PAM sequence positions the deaminase activity window (green) over the target C of E4 (green). Modified from Caso 2021. Panels E & F) Illustrates two base editor (BE) plasmid selection with selectivity for the PAM site for use in the base editing of ApoE4.

FIG. 2 illustrates synthetic exosomes with a cargo penetrating the blood brain barriers by squeezing through the tight junctions due to its deformability. Cargo-loaded SE (A) may enter the brain by (B) deforming to pass tight junctions of the vessel walls. SE/cargo may enter cells of the brain by endocytosis (C) or other mechanisms. In one illustrative, but non-limiting embodiment, we use a microfluidic based platform to encapsulate CNS biotherapeutic candidates in SEs. In certain embodiments, SEs for brain delivery are nanovesicles of ≤150 nm that are able to deform and cross the blood brain barrier while maintaining a cargo within.

FIG. 3, panels A-D illustrates a comparison of synthetic exosomes (SEs) to conventional liposomes (LPs) and natural exosomes (NEs). Atomic force microscopy (AFM) reveals (panel A) conventional LPs are not deformable, whereas (panel B) are compressed (deformable). Transmission electron microscopy (TEM) shows the morphological similarities between (panel C) NEs and (panel D) SEs.

FIG. 4, panels A-E, illustrates synthetic exosome synthesis, characterization, dialysis, and lyophilization. Panel A) Lipids (e.g., DPPC-1,2-dipalmitoyl-snglycero-3-phosphocholine, cholesterol, dihexadecyl phosphate (DCP); and (1-myristoyl-2-{P6-[(7-nitro-2-1,3-benzoxadizaol-4-yl)amino]helanoyl}-sj-glycero-3-phosphocholine) in IPA flow into an organic microfluidic reactor stream and cargo (if hydrophilic) in the aqueous stream. SEs are collected (panel B), characterized (panel C), dialyzed (panel D) and lyophilized for storage (panel E).

FIG. 5, panels A-I illustrate SE-sAPPα, SE-IDUA, SE_ASM and Se-Cas9-his deliver to mouse brain. Panel A) SE-sAPPα characteristics. sAPPα inhibition of BACE1 decreases (panel B) sAPPβ and (panel C) Aβ1-42 in CHO-7W cells. Panel D) sAPPα levels peak in brain 1 hour after SE-sAPPα delivery. Panel E) sAPPβ mean levels were lower with SE-sAPPα delivery than free sAPPα. ANOVA—Tukey's post-hoc analysis. Panel F) SE-(ξ+& −) IDUA characteristics. Panel G) Bran levels were highest with SE (ξ−)-IDUA. Panel H) ASM activity in brain were significantly higher after delivery with SE (ξ+)-ASM. ANOVA—Tukey's post-hoc analysis. Panel I) Brain levels of SE-Cas9-his by his tag detection were significantly higher with SE (ξ−)Cas9-his compared to free Cas9-his. ANOVA with Dunnett's comparison of SE to free Cas9-his. N=3/group; 2 for SE(+)Cas9his.

FIG. 6 indicates PAM site identification, editing window position, E4:E3 C:T target edit, and ARG:CYS change.

FIG. 7 illustrates a scheme for microfluidic SE-CRISPR synthesis and click chemistry for surface modification of SEs (Pep-1=SEQ ID NO:25; Pep-2=SEQ ID NO:26).

FIG. 8, panels A-C, illustrates a synthetic scheme, illustrative moieties for decorating synthetic exosomes (SEs), and an illustrative strategy for decoration of SE with peptides for enhanced brain permeability. A) Illustrative peptides for decorating synthetic exosomes (SEs). Panel B) Illustrative lipids for decorating synthetic exosomes (SEs). Panel C) Illustrative synthetic scheme for decorating SEs with peptides using a “click” chemistry.

### DETAILED DESCRIPTION

The results of several large clinical trials in Alzheimer's disease (AD) to date suggest that targeting amyloid-beta (Aβ) is not sufficient to stop disease progression and while newly approved aducanumab reduces Aβ pathology, it elicits only a modest improvement in cognition. An alternative approach would be to target apolipoprotein E4 (ApoE4, E4), the major genetic risk factor for sporadic AD [1-3] that, as compared to ApoE3, is associated with exacerbation of the two predominant hallmarks of AD brain—Aβ and tau pathology [4-11]. The risk conferred by the E4 gene may be decreased by converting it to E3 by use of CRISPR base editing technology, which allows editing in both dividing and non-dividing cells, critical for a significant effect in the brain. For gene editing to be possible in AD, CRISPR components must be delivered across the blood-brain barrier (BBB).

We have developed a brain delivery technology that involves microfluidic-reactor-based synthesis of deformable nanovesicles the size of natural exosomes (<150 nm), that we call “synthetic exosomes” (SEs) that encapsulate macromolecules. We have demonstrated that SE-encapsulated proteins, including CRISPR enzyme Cas9, can readily be delivered across the blood brain barrier to the mouse brain in sufficient quantity to provide effective CRISPR/cas mediated alterations to the genome of neural cells comprising the brain.

More specifically, in certain embodiments, synthetic exosomes (SEs) are provided that contain a gene editor (e.g., a B3 or B4 gene editor) and associated guide RNA (gRNA). The base editor plasmid to be used desirably has selectivity for the PAM site. It is believed such exosomes are able to deliver effective amount of the contained gene editor and/or gRNA across the blood brain barrier and into cells comprising the central nervous system. In various embodiments the SEs for brain delivery are liposomes of less than about 200 nm average (or median) diameter or less than about 150 nm average (or median) diameter that are able to deform while retaining a cargo within.

Without being bound to a particular theory, it is believed the synthetic exosomes are able to cross the blood-brain barrier (BBB) by physically squeezing between the tight junctions of the endothelial cells lining brain capillaries, as well as by the astrocytic projections (‘feet’) that also comprise the BBB, like miniature cells while protecting the therapeutic cargo encapsulated within the exosome.

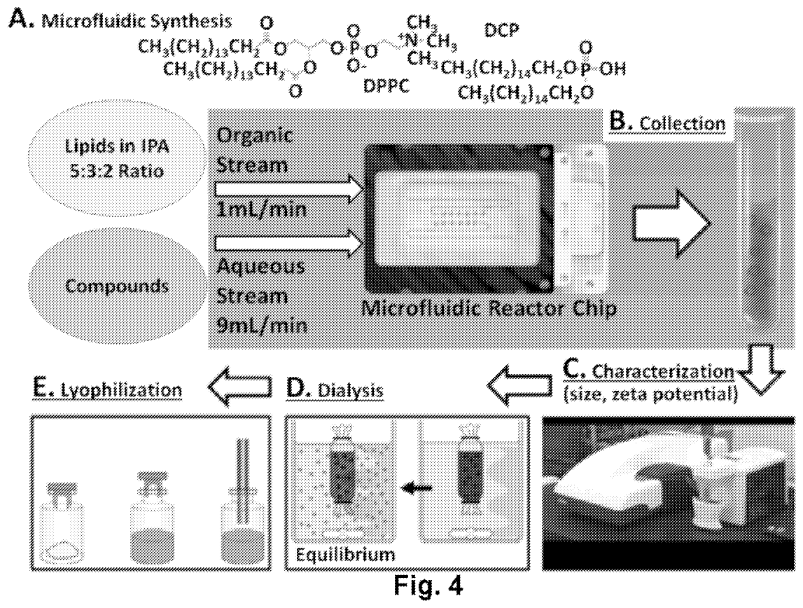

In various embodiments the therapeutic-loaded SEs are synthesized using a microfluidic flow reactor (see, e.g., FIG. 4). The SEs thus produced can be stored as a lyophilized powder for months without any loss of the drug cargo. In various embodiments the synthetic exosomes are composed of GRAS (generally regarded as safe) materials and are able to encapsulate and deliver large molecules such as a CRISPR gene editor and associated guide RNA (e.g., single guide RNA (sgRNA) across the blood brain barrier and into neural cells. In certain embodiments a variety of lipids such as DPPC (1,2-dipalmitoyl-snglycero-3-phosphocholine), cholesterol, and DCP (dihexadecyl phosphate) can be used in predetermined ratios to generate SEs with the desired properties including deformability.

The flow rates of the organic phase (typically carrying the lipids and lipophilic compounds) in, e.g., IPA (isopropyl alcohol) and aqueous phase (typically carrying the potential hydrophilic therapeutic molecules) can be finely controlled to yield SEs with specific size (60<φ<500 nm), zeta potential (−50<ξ<50) and deformability.

If desired the surface of the synthetic exosomes can be modified to include different surface charges, using a variety of molecules like PEG for longer circulatory half-life, and carrier proteins for targeted therapeutic delivery.

Having been optimized (as illustrated herein), the microfluidic synthesis of SEs is readily scalable for obtaining larger amounts and allows good batch-to-batch reproducibility.

As proof of principle, in our ongoing studies to achieve successful delivery of a potential therapeutic to the brain, we encapsulated a large protein fragment, the neurotrophic factor soluble Amyloid Precursor Protein alpha (sAPPα, 678 amino acids in length) and characterized the SE-sAPPα for size, encapsulation efficiency and zeta potential (see, e.g., Example 1).

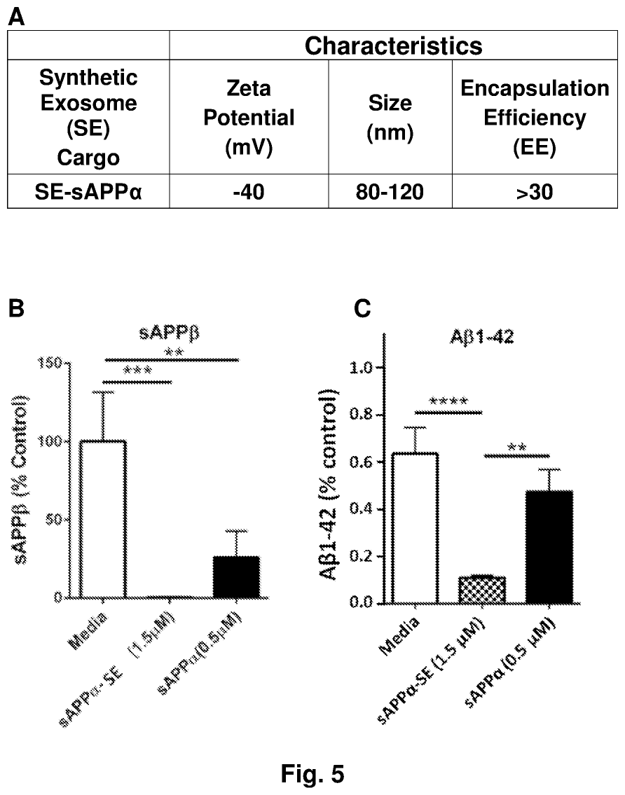

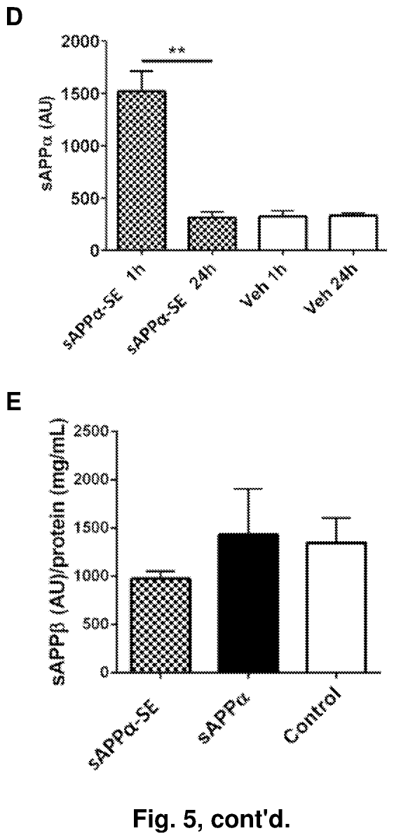

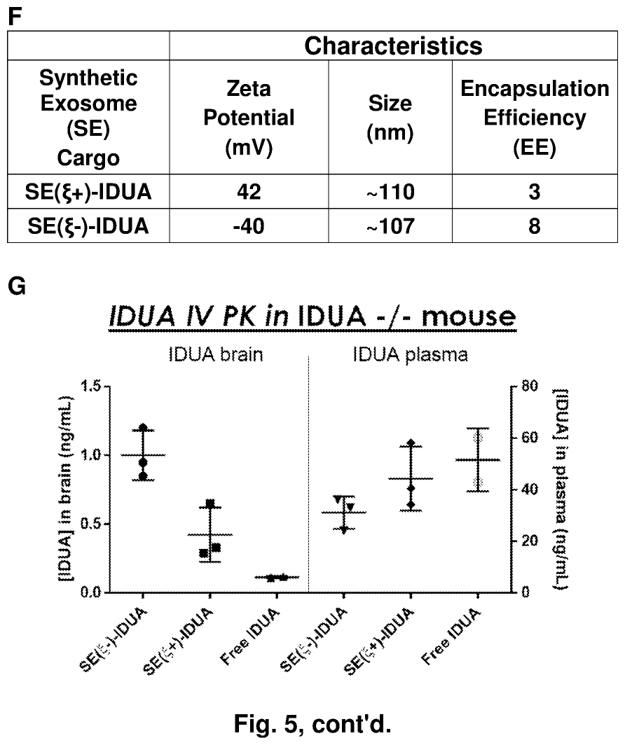

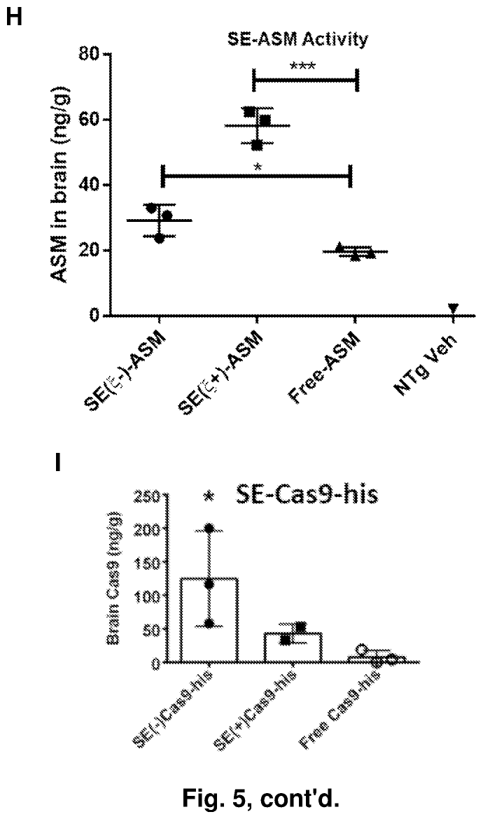

Next, to establish that we could achieve successful delivery and release of the SE payload to cells and maintain the biological activity of the cargo, we tested SE-sAPPα in vitro in cells. sAPPα is an endogenous inhibitor of BACE1 (beta-site cleaving enzyme 1), the enzyme responsible for cleavage of full-length (FL) APP resulting in production of sAPPβ and the β C-terminal fragment (βCTF). βCTF is then cleaved by the y secretase complex to produce amyloid-β (Aβ) and the APP intracellular domain. Therefore, successful delivery of sAPPα in cells that express FL APP should result in a decrease in sAPPβ and βCTF and, because of the decrease in the latter, a decrease in Aβ. As shown in Example 1 (see, e.g., FIG. 5) we found SE-sAPPα more significantly decreased sAPPβ as compared to the media only control than free sAPPα.

We also determined if peripheral injection of sAPPα-SEs would result in successful delivery of sAPPα to the brain of mice. To distinguish endogenously expressed sAPPα from the exogenous SE-encapsulated sAPPα, we used wildtype mice expressing only endogenous mouse APP and injected SEs loaded with recombinant human sAPPα; we then determined human sAPPα levels in brain using a human-sAPPα-specific AlphaLISA (Perkin-Elmer). As described in Example 1, after IV delivery of SE-sAPPα, levels of human sAPPα in mouse brain tissue were significantly higher at 24 hours. The results shown in Example 1 indicate successful brain delivery of potentially efficacious levels of sAPPα to the brain.

Finally, to ascertain target engagement, we injected SE-sAPPα into EFAD Alzheimer's disease (AD) model mice. EFAD transgenic mice are an AD model that express human apolipoprotein E4 (E) as well as human APP with three familial AD mutations and human presenilin 1 with two familial AD mutations (FAD); as a result, these mice show AD-like amyloid pathology at an early age, starting with increased production of sAPPβ. As shown in FIG. 5 sAPPβ was only lower than vehicle-only control with SE-sAPPα and not free sAPPα. This indicates target engagement and preservation of biological activity of sAPPα encapsulated in SEs. These data support the efficacy of SE-sAPPα delivery and the engagement of the target in the brain. Similarly, delivery of SE-IDUA in a knockout mice shows that the SEs deliver increased levels of IDUA into the brain. Additionally, delivery of another protein acid sphingomyelinase (ASM) that would be of benefit in the CNS disorder Niemann-Pick disease also shows increased activity in the brain in wild type mice after delivery in SE-ASM thus showing target engagement and brain delivery. Moreover, in view of these results it is believe the SEs described herein can be effectively utilized to deliver a gene editor (e.g., a cytidine base editor and associated guide RNA as described here) across the blood brain barrier and into cells of the central nervous system where the gene editor can alter target genes (e.g., a gene encoding ApoE4).

Accordingly, without being bound to a particular theory, it is believed that synthetic exosomes described herein containing a gene editor (e.g., B3 or B4 base editor) that targets and changes a gene encoding ApoE4 (e.g., changes the gene to one that encodes at least a part of ApoE3) finds use in the treatment and/or prophylaxis of a pathology implicating ApoE4. In certain embodiments such a pathology includes but is not limited to Alzheimer's disease (AD), frontotemporal dementia, cerebral amyloid angiopathy (CAA), dementia with Lewy bodies (DLB), tauopathy, cerebrovascular disease, multiple sclerosis, vascular dementia, and treatment after traumatic brain injury and/or hemorrhage.

Base-Editor for Treatment and/or Prophylaxis of a Neurological Pathology.

We have developed Synthetic Exosomes (SEs), microfluidic-reactor synthesized deformable nanovesicles the size of natural exosomes, as a platform to deliver macromolecules across the blood-brain barrier (BBB) and as described herein, CRISPR components (e.g., SE-CRISPR) can be incorporated to provide genetic editing, e.g., of ApoE4 a major genetic risk factor for Alzheimer's disease and certain other pathologies, e.g., as described herein.

In certain embodiments the SEs described herein contain a cytidine base editor. Typical cytidine base editors (CBEs), comprise a cytidine deaminase fused to Cas9 nickase (nCas9) and such base editors enable efficient C-to-T conversion in various organisms.

Typically such base editing utilizes a Cas nickase or Cas fused to a deaminase that makes the edit, a guide RNA (gRNA) targeting the Cas to a specific locus, and a target base for editing within the editing window specified by the Cas protein.

These elements were the starting point towards the development of the first cytosine base editors as described Komor et al. (2016) Nature, 533: 420-424. Komor et al. (Id.) created the first cytosine base editor by coupling a cytidine deaminase with the inactive dCas9 (Id.). These fusions convert cytosine to uracil without cutting DNA. Uracil is then subsequently converted to thymine through DNA replication or repair. Fusing an inhibitor of uracil DNA glycosylase (UGI) to dCas9 prevented base excision repair which changes the U back to a C mutation. To increase base editing efficiency a mechanism can be used to force the cell to use the deaminated DNA strand as a template. To do so, Komor et al. (Id.) used a Cas nickase, instead of dCas9. The resulting editor, often designated BE3, nicks the unmodified DNA strand so that it appears “newly synthesized” to the cell. Thus, the cell repairs the DNA using the U-containing strand as a template, copying the base edit.

The BE3 system increased editing frequency to above 30% for a variety of targets in human cell lines, with an average indel frequency of only 1.1%. These numbers are a vast improvement over Cas9-mediated HDR for the loci tested where average HDR-mediated editing frequency was only 0.5%, and more indels were observed than point modifications. CRISPR base editing persists through multiple cell divisions, indicating that this method produces stable edits. However, this system can also subject to off-target editing based on Cas9 off-target activity.

Since the development of BE3, subsequent improvements to base editors include but are not limited to 1) Expanded target scope; 2) Improved editing efficiency; and 3) Decreased off-target effects.

Thus, for example, the Target-AID base editor uses acytidine deaminase from sea lamprey fused to Cas9 nickase (see, e.g., Nishida et al. (2016) Science 353: aaf8729-aaf8729). Target-AID acts similarly but not identically to BE3, modifying a 3-5 base window 18 bases upstream of the PAM.

BE3 variants with other deaminases, e.g., AID, CDA1, and APOBEC3G have also been created (see, e.g., Komor et al. (2017) Sci. Adv. 3: eaao4774; Komor et al. (2017) Nature. 551: 464-471; and the like). CDA1-BE3 and AID-BE3 edited Cs following a G more efficiently than BE3, but APOBEC3G displayed less predictable sequence preferences.

Additionally, natural and engineered Cas9 variants have been used to develop new base editors with distinct PAM sequences, expanding the number of available target sites for base editing (see, e.g., Kim et al. (2017) Nat. Biotechnol. 35: 371-376). For each base editor, they observed editing activity with a minimum efficiency of ˜50% and confirmed that the fusion protein retained the PAM properties of the individual Cas9. They also mutagenized the cytidine deaminase portion of the base editor to create SpCas9 base editors with editing windows as small as 1-2 nucleotides.

To reduce off-target effects associated with base editing, HF-BE3, a base editor containing high fidelity Cas9 variant HF-Cas9 has been developed (see, e.g., Rees et al. (2017) Nat. Commun. 8: 15790). HF-BE3 showed 37-fold less off-target editing than BE3, with only a slight reduction in on-target editing efficiency. To further improve specificity, they purified HF-BE3 protein for delivery in ribonucleoprotein particles (RNPs) to both zebrafish embryos and the mouse inner ear.

The fourth-generation base editors, BE4, reduce the undesired C->G or C->A conversions that can happen with BE3's. It is believed these byproducts result from excision by uracil N-glycosylase (UNG) during base excision repair. Adding a second copy of the UNG inhibitor, UGI, increases base editing product purity. Additionally, the APOBEC1-Cas9n and Cas9n-UGI linkers have been extended to improve product purity, and these three improvements represent the fourth generation of base editors. Compared to BE3, BE4 can offer a 2.3-fold decrease in C->G and C->A products as well as a 2.3-fold decrease in indel formation.

To further decrease indel formation 1.5-2-fold, bacteriophage protein Gam has been fused to the N-terminus of BE4 (see, e.g., Komor et al. (2017) supra.). Gam binds the free ends of DSBs, which may lead to cell death rather than NHEJ repair, thus removing these cells from the edited population.

Another way to improve base editing efficiency for mammalian edits is to facilitate entry of the editor into the nucleus. To accomplish this, nuclear localization signals and codon usage has been modified in BE4 to create BE4max and AncBE4max with a 4.2-6-fold improvement in editing efficiency (see, e.g., Koblan et al. (2018) Nat. Biotechnol. 36: 843-846).

It will be recognized that any of these, and numerous other base editors can be incorporated into the synthetic exosomes described herein to target and edit a gene encoding ApoE4.

Apolipoproteins E3 and E4, proteins with a molecular mass of 34.15 kDa, differ by a single amino acid change. In particular, ApoE4 contains an arginine residue at position 112, whereas apoE3 has a cysteine at this position. ApoE4 is the major risk factor for late-onset Alzheimer's disease, whereas apoE3, the common isoform, is neutral with respect to this disease.

Accordingly, in various illustrative, but non-limiting embodiments, the gene editor contained in the SEs described herein targets and edits one or more codons found in ApoE4. In certain embodiments the gene editor targets ApoE4 codon 112 and converts it to the corresponding codon in ApoE3 encoding cysteine.

We can demonstrate proof-of-concept for the feasibility of E4 to E3 gene editing by IV administration of SE-CRISPR, optimized for brain permeability and E4 to E3 gene editing efficiency, to E4 targeted-replacement (E4TR) mice. We can assess E4 to E3 gene editing in mouse brain, by cell type (neurons, microglia, astrocytes, etc.) and by region (hippocampus, etc.) using Next-Generation Sequencing (NGS); and E4 and E3 protein levels by mass spectrometry.

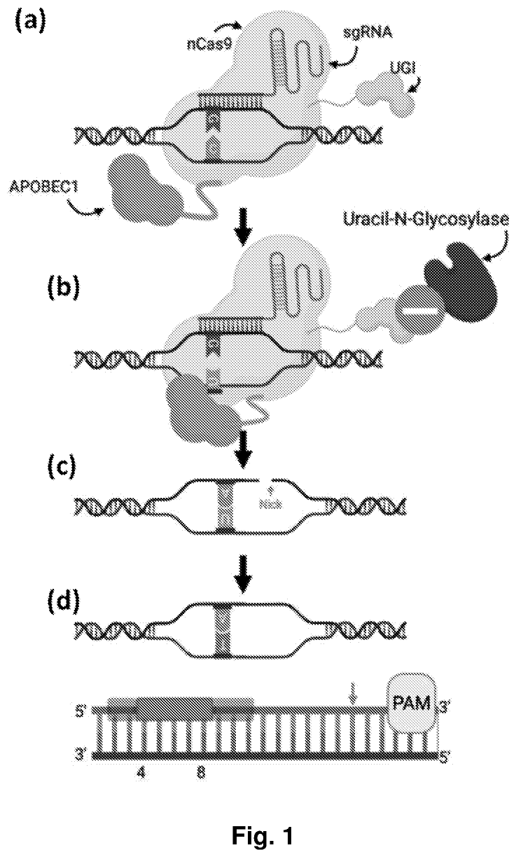

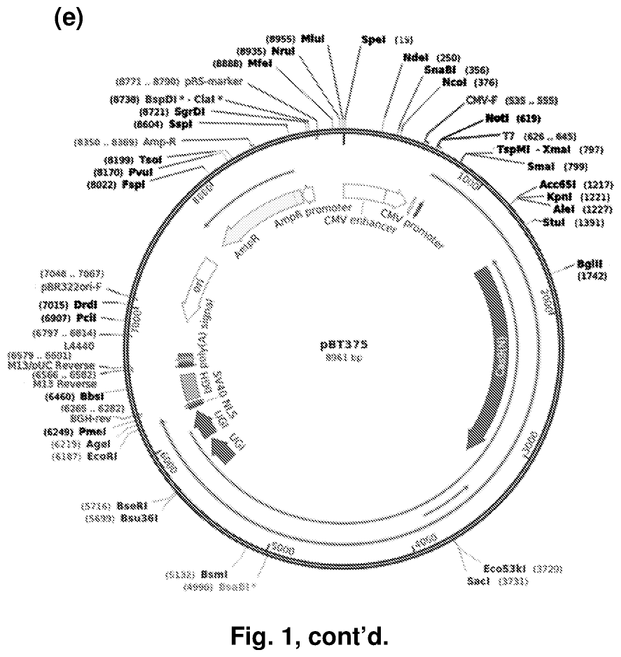

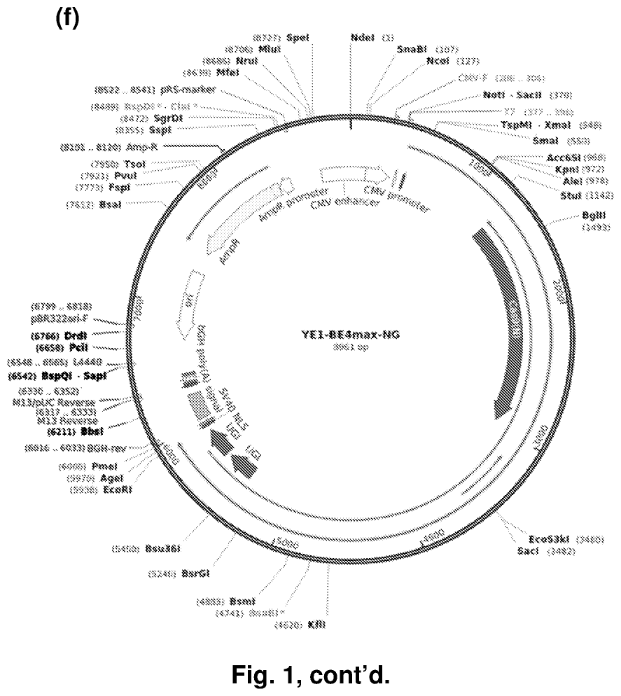

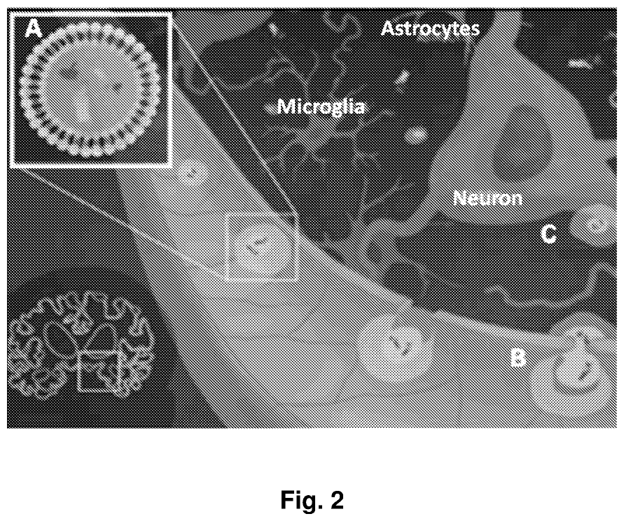

In certain embodiments a base editor tool (e.g., B3, B4, BE4max, AncBE4max, and the like) to convert E4 to E3. As shown in FIG. 1, base editing (BE) allows direct, irreversible conversion of a single DNA nucleobase into another without requiring double-stranded DNA (dsDNA) breaks. CRISPR/Cas9 and cytidine deaminase enzyme fusion proteins are used to effect the substitutions, leading to correction of ˜15-75% of total cellular DNA with minimal (≤1%) INDEL formation. Important for efficient E4 to E3 conversion in the brain, BE can be used for non-dividing, as well as dividing, cells [38]. E4 edited to E3 is predicted to not only affect the edited cell but neighboring cells as well, because ApoE produced by glial cells in the brain is secreted and then taken up by other cells via receptor-mediated endocytosis [27]. While the use of base editors (BE) is described herein, it will be recognized that in certain embodiments, prime editing (PE) that utilizes “search-and-replace” genome editing and that can rewrite all 12 possible base conversions is another possible approach.

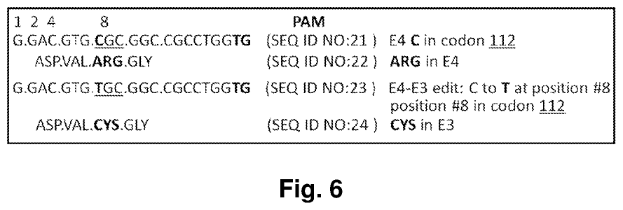

As noted above the many advantages of base editing include increased efficiency (>20% to 80%) and reduced INDELs (<1%); its disadvantages are precise PAM site spacing and bystander editing [36, 37, 39]. Base editing requires precise positioning of the Cas9-linked deaminase by gRNA over the target DNA site (see FIG. 1) which abuts a nearby PAM sequence. We analyzed the E4 sequence and identified a candidate PAM (see, e.g., FIG. 6, and Table 1). As shown therein, the E4 sequence of the protospacer sequence numbering relative to PAM (TG) includes the deaminase peak activity window, which is from base 4 to 8, but peripheral deaminase activity can extend from base 2 to 11. In Komor et al. [30], they demonstrated that the E4 C in codon 112 can be deaminated in vitro. The deaminase activity is a 5 bp window, so other bystander Cs can be deaminated and would be evaluated for mutation effects. All possible deaminations of bystander Cs, result in silent mutations; the C at position 4 in codon 110 (GAC to GAT) code for asparagine; and both Cs at 8 and 10 (outside peak activity window; TGC to TGT) code for cysteine. Other potential C bystanders at 10 and 13, outside peak or peripheral activity windows, encode arginine and glycine, respectively.

In one illustrative embodiment, the gRNA is the same 20 nt sequence as the protospacer sequence abutting the PAM. This gRNA would be synthesized (Synthego) with an 80 nt tracrRNA that specifically binds Cas9 protein (“sgRNA” in FIG. 1). Importantly, the sequence of this region of E4 can verified by Sanger sequencing in both the models used: the N2a-E4 cells and E4 TR mice.

The BE tool Cas9 is a fusion of 3 proteins (FIG. 1), with BE4 being the latest generation [36, 37, 39]. The Cas9 PAM recognition activity will be for the TG PAM site designed above, specifically SpCas9n-NG [44]. We can use a Cas9 bacterial expression vector to produce bulk protein, and this can be combined with gRNA in a BE:gRNA complex for encapsulation in SEs.

To optimize the BE-gRNA, its editing activity will be predicted in silico using BE-Hive [45, 46] and validated in vitro by electroporation of the BE:gRNA complex into N2a-E4 cells using different ratios of components. The negative control will not have gRNA. Genomic DNA will be PCR amplified, Sanger sequenced with high quality results, and base-editing efficiency (on target and bystander) quantified using EditR [47]. Sanger/EditR is valid for >10% efficiency; for <10%, NGS (using Amplicon-EZ (Genewiz.com)) at 50K reads (using the fewest possible PCR cycles) and analyzed using CRISPResso2 [48]. BE:gRNA combination optimization balances on-target efficiency with undesired INDELs around the E4 site and off-target editing. INDELs will also be quantified from Sanger (sensitivity limit >5%) using ICE [43]. The 10 top most probable sites for gRNA-dependent off-target effects will be predicted using Cas-OFFinder [49], then each site will undergo PCR amplification followed by Sanger sequencing and ICE analysis.

We expect at least moderate efficiency (>20%) with infrequent off-target editing. Once the BE and gRNA are validated, large-scale synthesis of gRNA (Synthego) and the Cas9/BE (using bacterial expression bulk production at UCLA core) will generate sufficient material for SE encapsulation for in vitro and in vivo studies.

TABLE 1
Illustrates one embodiment showing the E4 sequence around the C in codon 112 is
shown, with the PAM site and its upstream protospacer region and the deaminase
activity window at positions #2 to #11.

| | | SEQ |
|---|---|---|
| Sequence | Comment | ID NO |
| Protospacer region numbering relative to PAM | | |
| deaminase activity window is from #2 to #11 | codon 112, E4 C | 1 |
| 1 11 PAM | ARG in E4 | 2 |
| GGAG.GAC.GTG.CGC.GGC.CGCCTGG | | |
| ASP.VAL.ARG.GLY | | |
| desired E4-E3 edit in 112: C at position #11 | codon 112, E3 T | 3 |
| inside activity window | CYS in E3 | 4 |
| 1 11 PAM | | |
| GGAG.GAC.GTG.TGC.GGC.CGCCTGG | | |
| ASP.VAL.CYS.GLY | | |
| undesired edit in codon 110: C at position #7 | codon 110, C-T: | 5 |
| inside activity window | GAC and GAT are both ASP | 6 |
| 1 7 PAM | | |
| GGAG.GAT.GTG.CGC.GGC.CGCCTGG | | |
| ASP.VAL.ARG.GLY | | |

It will be recognized that alternative PAMs and gRNA/protospacer sequences can be exploited. One such illustrative embodiment is illustrated in Table 2 which also shows the predicted bystander edits.

TABLE 2
Illustrates alternative PAM placing 112 C within peak activity window

| | | SEQ |
|---|---|---|
| | | ID |
| Sequence | Comment | NO |
| Alternative PAM placing 112 C within peak activity window | | |
| Protospacer region numbering relative to PAM, | codon 112, E4 C | 7 |
| deaminase peak activity window is from #4 to #8 | ARG in E4 | 8 |
| 1 8 PAM(NG) | | |
| GGAG.GAC.GTG.CGC.GGC.CGCCTGGTGCAGTACCGCGG | | |
| ASP.VAL.ARG.GLY | | |
| desired E4-E3 edit in 112: C at position #8 inside activity | codon 112, E3 T | 9 |
| window | CYS in E3 | 10 |
| 1 8 PAM(NG) | | |
| GGAG.GAC.GTG.TGC.GGC.CGCCTGGTGCAGTACCGCGG | | |
| ASP.VAL.CYS.GLY | | |
| Bystander Edits | | |
| 1. desired E4-E3 edit in 112: both Cs at positions #8 (in | codon 112, E4 C | 11 |
| activity window) and #10 (outside peak activity window) | TGC and TGT | 12 |
| 1 8 10 PAM(NG) | are both CYS in | |
| GGAG.GAC.GTG.TGT.GGC.CGCCTGGTGCAGTACCGCGG | E3 | |
| ASP.VAL.CYS.GLY | | |
| 2. undesired edit in codon 110: C at position #4 inside activity | codon 110 | 13 |
| window | GAC and GAT | 14 |
| 1 4 PAM(NG) | are both ASP | |
| GGAG.GAT.GTG.CGC.GGC.CGCCTGGTGCAGTACCGCGG | | |
| ASP.VAL.ARG.GLY | | |
| 3. undesired E4-E3 edit in 112: C at position #10 is in the non- | codon 112, E4 C | 15 |
| peak activity window, rarely deaminated | CGC and CGT | 16 |
| 1 10 PAM(NG) | are both ARG in | |
| GGAG.GAC.GTG.CGT.GGC.CGCCTGGTGCAGTACCGCGG | E4 | |
| ASP.VAL.ARG.GLY | | |
| 4. undesired edit in codon 113: C at position #13 is outside | | |
| activity window and even if rarely deaminated produces a silent | | |
| mutation (GLY) | | |

In certain illustrative, but non-limiting embodiments the sequence of the “gRNA” that guides the Cas9 nuclease-deaminase fusion protein to the apoE4 gene DNA site has three parts that are linked into a single RNA molecule:
1) a crRNA;
2) a tracrRNA; and
3) a linker region RNA.

In certain embodiments the CRSPR RNA (crRNA) comprises or consists of a 20nt RNA specific for the APOE4 gene near the nucleotide position that is to be edited (e.g., sequence: GGACGUGCGCGGCCGCCUGG (SEQ ID NO:17)). In certain embodiments the trans-activating CRISPR RNA (tracrRNA) is an 80 nt RNA that provides the “stem loop” structure bound by the CRISPR nuclease (e.g., Cas9). Suitable linker regions connecting the tracrRNA to the crRNA and tracrRNA are commercially available from Synthego Corp. (Redwood City, CA). One illustrative, but non-limiting example of a suitable tracrRNA is the tracrRNA for wildtype SpCas9 (Streptococcus pyogenes Cas9), that was used to discover modifications that improved the tracrRNA activity (see, e.g., Scott et al. (2019) Sci Rep. 9: 16104). One illustrative sequence for a SpCas9 tracrRNA comprises the sequence AGCAUAGCAAGUUUAAAUAAGGCUAGUCCGUUAUCAACUUGAAAAAGUGGCA CCGAGUCGGUGCUUU(SEQ ID NO:18).

It will be recognized that the above-illustrated gene editor constructs and protospacer/gRNA sequence are illustrative and non-limiting. Using the teaching provided herein alternative gene editor constructs and protospacer/gRNA sequences will be available to one of skill in the art.

### Synthetic Exosome Formulations for Delivery of CRISPR/Cas Constructs to the Brain.

In various embodiments the synthetic exosomes described herein comprise a liposome formed from a lipid bilayer, where the lipid bilayer comprises or consists of:
A) one or more phospholipids selected from the group consisting of phosphate lipids, phosphoglycerol lipids, phosphocholine lipids, and phosphoethanolamine lipids where the lipid carbon chain ranges from 3 to 24 carbon atoms;
B) cholesterol, cholesterol hemicsuccinate, or a phytosterol; and
C) a non-ionic surfactant.

In various embodiments, the synthetic exosome contains a gene editor (e.g., a CRISPR gene editor such as a cytidine base editor (e.g., n-Cas9-cytidine deaminase, and an associated guide RNA (e.g., gRNA).

In certain embodiments the synthetic exosomes range in size from about 50 nm up to about 200 nm in diameter. Typically, the synthetic exosomes are capable of crossing the blood brain barrier without substantially leaking the therapeutic moiety (gene editor and/or guide RNA). In certain embodiments the exosomes are capable of crossing the blood/brain barrier (BBB) and delivering the therapeutic moiety (gene editor and/or guide RNA) contained therein to the central nervous system without losing more than about 40%, or without losing more than 30%, or without losing more than 20%, or without losing more than 10%, or without losing more than 5%, or without losing more than 3%, or without losing more than 1% of the gene editor and/or guide RNA contained therein.

In certain embodiments the lipid bilayer comprising the synthetic exosomes consists of one or more phospholipids (e.g., 1, 2, 3, 4, or more phospholipids), cholesterol and/or cholesterol hemisuccinate, and/or a phytosterol; and a non-ionic surfactant.

In certain embodiments the lipid bilayer comprising the synthetic exosomes does not contain an alcohol (e.g., ethanol). In certain embodiments the lipid bilayer comprising the synthetic exosomes does not contain glutathione-maleimide-PEG2000-distearoyl phosphatidyl ethanolamine In various embodiments the synthetic exosomes are not transferosomes or ethosomes.

In certain embodiments the molar ratio of total phospholipid to cholesterol, cholesterol hemisuccinate, and/or phytosterol ranges from about 4-8 moles of phospholipids to about 1-2 moles of cholesterol.

In certain embodiments the amount of surfactant ranges from about 1%, or from about 3%, or from about 5%, or from about 8% up to about 18%, or up to about 15%, or up to about 13%, or up to about 10% (wt/wt). In certain embodiments the surfactant comprises one or more surfactants selected from the group consisting of Span 80, Tween 20, BRIJ® 76 (stearyl poly(10)oxy ethylene ether), BRIJ® 78 (stearyl poly(20)oxyethylene ether), BRIJ® 96 (oleyl poly(10)oxy ethylene ether), and BRIJ® 721 (stearyl poly (21) oxyethylene ether). In various embodiments the surfactant comprises or consists of Span 80.

In certain embodiments the synthetic exosome lipid bilayer (LB) comprises about 10% to about 20%, or about 15% Span 80 by weight.

### Phospholipids

In various embodiments the lipid bilayer comprising the synthetic exosomes described herein is formed from 1, 2, 3, or 4, or more phospholipids, cholesterol or a functionalized cholesterol (e.g., cholesterol hemisuccinate (CHEMS), lysine-based cholesterol (CHLYS), and PEGylated cholesterol (Chol-PEG), or a phytosterol, and one or more surfactants (e.g., Span-80).

In certain embodiments the synthetic exosomes bear one or more targeting moieties attached to the lipid bilayer. Illustrative targeting moieties include, but are not limited to amino acids (e.g., amino acid functionalized lipids) that are transported into the central nervous system (CNS) by amino acid transporters, transferrin folic acid, various antibodies, CD171, and the like.

In various embodiments the lipid bilayer comprises one or more phospholipids, including, but not limited to phosphate, phosphoglycerol and phosphocholine lipids, phosphoethanolamine lipids with/without polyethylene glycol (7-100 monomers), and cholesterol. In various embodiments the lipid carbon chain ranges from about 3 to about 24 carbons atoms. Suitable phospholipids for use in the synthetic exosomes described herein can include Dihexanoyl-sn-glycero-3-phosphate (DHPA), Didecanoyl-sn-glycero-3-phosphate (DDPA), Distearoyl-sn-glycero-3-phosphate (DTPA), Dihexadecyl phosphate (DHP), and the like.

Phosphoglycerol lipids suitable for use in the lipid bilayer comprising the synthetic exosomes described herein can include but are not limited to Dihexanoyl-sn-glycero-3-phospho-(1′-rac-glycerol) (DHPG), Dilauroyl-sn-glycero-3-phospho-(1′-rac-glycerol) (DLPG), Distearoyl-sn-glycero-3-phospho-(1′-rac-glycerol) (DTPG), and the like.

Phosphocholine lipids suitable for use in the lipid bilayer comprising the synthetic exosomes described herein can include but are not limited to Dipropionyl-sn-glycero-3-phosphocholine (PC), Diheptanoyl-sn-glycero-3-phosphocholine (DHPC), Dimyristoyl-sn-glycero-3-phosphocholine (DMPC), Dilignoceroyl-sn-glycero-3-phosphocholine (DGPC), and the like.

Phospholipids with an alkyne such as phosphoethanolamine suitable for use in the lipid bilayer comprising the synthetic exosomes described herein can include but are not limited to Dihexanoyl-sn-glycero-3-phosphoethanolamine (DHPE), Distearoyl-sn-glycero-3-phosphoethanolamine (DTPE), and the like.

Phosphoethanolamine-PEG lipids suitable for use in the lipid bilayer comprising the synthetic exosomes described herein can include but are not limited to Dipalmitoyl-sn-glycero-3-phospho(ethylene glycol) (DPPEG1), Dimyristoyl-sn-glycero-3-phosphoethanolamine-N-[methoxy(polyethylene glycol)-350 (DMPEG350), Distearoyl-sn-glycero-3-phosphoethanolamine-N-[methoxy(polyethylene glycol)-350] (DTPEG350), Dimyristoyl-sn-glycero-3-phosphoethanolamine-N-[methoxy(polyethylene glycol)-550] (DMPEG550), Dimyristoyl-sn-glycero-3-phosphoethanolamine-N-[methoxy(polyethylene glycol)-1000] (DMPEG1000).

Other lipids suitable for use in the lipid bilayer comprising the synthetic exosomes described herein can include but are not limited to Dioleoyl-sn-glycero-3-phosphocholine (N-aminoethyl) (PC-NH2), Diphytanoyl-sn-glycero-3-phosphoethanolamine, Dioleoyl-3-trimethylammonium-propane (DOTAP), Distearoyl-3-trimethylammonium-propane (DSTAP), Dimyristoyl-3-trimethylammonium-propane (DMTAP), Di-O-octadecyl-sn-glycero-3-phosphocholin (DOPC), and the like.

### Cholesterol and Cholesterol Analogs/Derivatives

In various embodiments the lipid bilayer comprising the synthetic exosomes described herein contains cholesterol, a cholesterol derivative, or a cholesterol analog (e.g., a phytosterol).

Illustrative cholesterol derivatives include, but are not limited to cholesterol hemisuccinate, lysine-based cholesterol (CHLYS), 20-hydroxychloesterol, 22-hydroxycholesterol, 24-hydroxycholesterol, 25-hydroxy cholesterol, 27-hydroxycholesterol, cholesteryl succinate, cholic succinate, cholic tri-succinate, lithocholic succinate, chenodesoxycholic bis-scuccinate, Hederoside, and the like. In certain embodiments the cholesterol derivative is cholesterol hemisuccinate.

In certain embodiments a phytosterol is used in addition to cholesterol, or in place of cholesterol. Suitable phytosterols include but are not limited to 9,10-secosteroids (e.g., vitamin D3, vitamin D2, calcipotriol, and the like), C-24 alkyl steroids (e.g., stigmasterol, β-sitosterol, and the like), and pentacyclic steroids (e.g., betulin, lupeol, ursolic acid, and oleanolic acid). In certain embodiments the phytosterol is a C-24 alkyl steroid.

It will be recognized that whenever cholesterol (CHOL) is described herein the cholesterol, cholesterol derivative, or phytocholesterol is further functionalized. Thus, for example, in certain embodiments, the cholesterol, cholesterol derivative, or phytocholesterol is pegylated.

### Particular Effective Synthetic Exosome Formulations.

The particular lipid mixture used for a synthetic exosome is determined by consideration of the nature of the molecule(s) to be entrapped, the anatomical delivery location and the desire surface charge. For example, for biological molecules (e.g., gene editors and guide RNAs, and the like) a mixture of 2-4 lipids with small carbon chains (e.g., 3-14 carbon atoms), can be used in combination with cholesterol, and/or functionalized cholesterol, and/or phytosterol, and a surfactant (e.g., Span-80) are utilized to form the exosome. In various embodiments illustrative, but non-limiting embodiments, the concentration of surfactant (e.g., Span-80) can range from about 1%, or from about 5% up to about 20%, or up to about 15% (w/w) depending on the degree of deformability needed. Illustrative formulations particularly well suited for delivery of biologic payloads are shown in Table 3.

TABLE 3
Illustrative synthetic exosome formulations for
delivlery of nCas9-cytidine deaminase and gRNA.

| Zeta | Lipid mixtures (in equimolar) so |
|---|---|
| Potential (ζ) | ratios below are molar ratios. |
| (mV) | Example for biologics |
| −20 ≤ ζ | 3:2:1 (DHPA:DHP:CH) |
| | 1:5:1 (DHPG:DHPA:CH) |
| | 2:5:1:2 (DHPG:DHPA:PC—NH2:CH) |
| | 2:4:1:1:2 (DHPG:DHPA:PC—NH2:DMPEG350:CH) |
| −20 ≤ ζ ≤ 20 | 2:2:1 (DHPG:DHPC:CH) |
| | 4:4:1:2 (DHPG:DHPA:PC—NH2:CH) |
| | 2:2:1 (DHPG:DHPA:CH) |
| | 4:4:1:1:2 (DHPG:DHPA:PC—NH2:DMPEG550:CH) |
| | 2:2:1 (DHPG:DTPE:CH) |
| | 2:2:1 (DHPG:DMTAP:CH) |
| | 4:4:1:2 (DHPG:DMTAP:PC—NH2:CH) |
| | 4:4:1:1:2 (DHPG:DMTAP:PC—NH2:DMPEG550:CH) |
| Z > 20 | 2:4:1 (DHPC:DTPE:CH) |
| | 2:4:1 (DHPC:DOTAP:CH) |
| | 2:4:1:2 (DHPC:DMTAP:PC—NH2:CH) |
| | 2:4:1:1:2 (DHPC:DMTAP:PC—NH2:DMPEG350:CH) |

*In certain embodiments, CH is cholesterol. In certain embodiments, CH is a cholesterol derivative (e.g., CHEMS, CHLYS, Chol-PEG, and the like). In certain embodiments CH is a phytosterol (e.g., 9,10-secosteroids (e.g., vitamin D3, vitamin D2, calcipotriol, and the like), C-24 alkyl steroids (e.g., stigmasterol, β-sitosterol, and the like), and pentacyclic steroids (e.g., betulin, lupeol, ursolic acid, and oleanolic acid).

In certain embodiments, CH in the formulations in Table 3 is cholesterol.

In certain embodiments, CH in the formulations in Table 3 is a cholesterol derivative. Thus, in certain embodiments, CH in the formulations in Table 3 is cholesterol hemisuccinate. In certain embodiments, CH in the formulations in Table 3 is lysine-based cholesterol (CHLYS). In certain embodiments, CH in the formulations in Table 3 is 20-hydroxychloesterol. In certain embodiments, CH in the formulations in Table 3 is 22-hydroxycholesterol. In certain embodiments, CH in the formulations in Table 3 is 24-hydroxycholesterol. In certain embodiments, CH in the formulations in Table 3 is 25-hydroxy cholesterol. In certain embodiments, CH in the formulations in Table 3 is 27-hydroxycholesterol. In certain embodiments, CH in the formulations in Table 3 is cholesteryl succinate. In certain embodiments, CH in the formulations in Table 3 is cholic succinate. In certain embodiments, CH in the formulations in Table 3 is cholic tri-succinate. In certain embodiments, CH in the formulations in Table 3 is lithocholic succinate. In certain embodiments, CH in the formulations in Table 3 is chenodesoxycholic bis-scuccinate. In certain embodiments, CH in the formulations in Table 3 is Hederoside.

In certain embodiments, CH in the formulations in Table 3 is a phytosterol. Thus, in certain embodiments CH in the formulations in Table 3 is a 9,10-secosteroids. In certain embodiments CH in the formulations in Table 3 is vitamin D3. In certain embodiments CH in the formulations in Table 3 is vitamin D2. In certain embodiments CH in the formulations in Table 3 is calcipotriol. In certain embodiments CH in the formulations in Table 3 is a C-24 alkyl steroid. In certain embodiments CH in the formulations in Table 3 is stigmasterol. In certain embodiments CH in the formulations in Table 3 is β-sitosterol. In certain embodiments CH in the formulations in Table 3 is a pentacyclic steroid. In certain embodiments CH in the formulations in Table 3 is betulin. In certain embodiments CH in the formulations in Table 3 is lupeol. In certain embodiments CH in the formulations in Table 3 is ursolic acid. In certain embodiments CH in the formulations in Table 3 is oleanolic acid.

The foregoing formulations are illustrative and non-limiting. Using the teaching provided herein, numerous other exosome formulations for the deliver of nCas9-cytidine deaminase and a gRNA to cells of the central nervous system will be available to one of skill in the art.

### Targeting Moieties

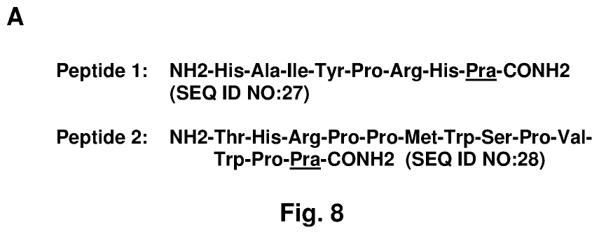

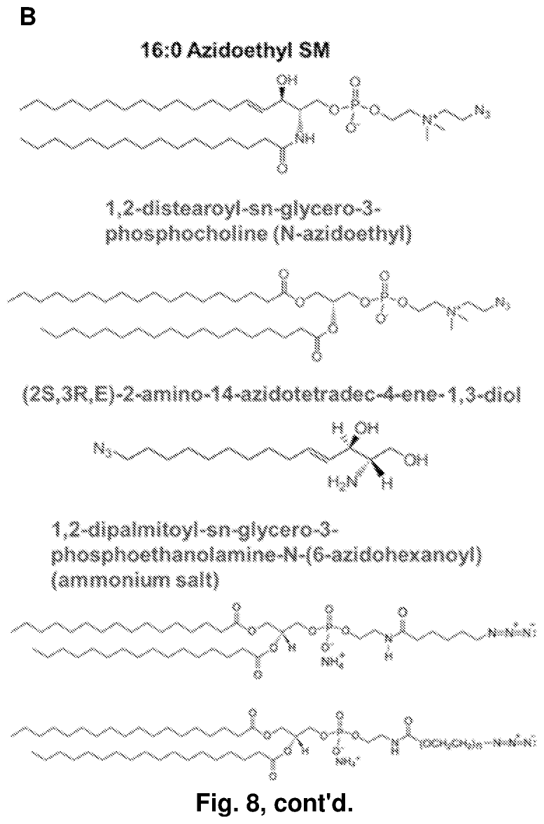

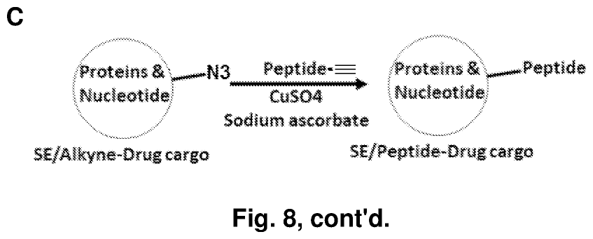

In certain embodiments the synthetic exosomes bear one or more targeting moieties attached to the lipid bilayer. Illustrative targeting moieties include, but are not limited to amino acids (e.g., amino acid functionalized lipids) that are transported into the central nervous system (CNS) by amino acid transporters, transferrin (e.g., transferrin functionalized lipids), transferrin receptor binding peptides (see, e.g., FIG. 8, panel A), folic acid, various BBB endothelial cell binding antibodies, CD171, and the like.

In certain embodiments the phospholipid or cholesterol comprising the synthetic exosomes described herein can be functionalized to thereby attach one or more targeting moieties. In certain embodiments the targeting moieties can comprise an amino acid to exploit amino acid transporters for internalization into a target cell. Essential amino acids are commonly transported across the BBB through specific transporters to participate in brain amino acid metabolism, such as the synthesis of neurotransmitters. Based on the difference of the substrates, amino acid transporters are divided into cationic, anionic, and neutral amino acid transporters. Large neutral amino acid transporter (LAT1) is the most abundant carrier for amino acids, which is expressed on both luminal and abluminal membranes of BCECs. LAT1 carries large neutral amino acids such as leucine, tryptophan, tyrosine, and phenylalanine across the BBB in the ion-independent pathway. Accordingly, in certain embodiments the targeting moiety can comprise carries a large neutral amino acid such as leucine, tryptophan, tyrosine, and phenylalanine.

Other illustrative targeting moieties include, but are not limited to antibodies, lectins, transferrin, folic acid, CD171, and the like.

Methods of coupling targeting moieties to liposomes and by extension the synthetic exosomes are well known to those of skill in the art. In certain embodiments the SEs described here are coupled to targeting moieties utilizing a click chemistry, e.g., as illustrated in FIG. 8. As shown in FIG. 8, at room temperature and in the presence of CuSO4, phosphate-terminal azides displayed on the surface of SEs synthesized using lipid azides undergo a cycloaddition reaction to form covalent bonds with the propargylglycine (Pra) amino acid of transferrin (Tf) peptides or PEG-alkyne for adding PEGs. The yield of the click reaction is very high. In FIG. 8, Tf peptide with either 8 or 13-amino acids or 2-PEG moieties (PEG2) or (PEG24) are shown.

### Pegylation.

In certain embodiments the synthetic exosomes described herein are pegylated (e.g., functionalized with a polyethylene glycol (PEG). Methods of pegylating liposomes are well known to those of skill in the art. In certain embodiments pegylation is achieved by incorporating one or more PEG-conjugated phospholipids into the lipid bilayer forming the synthetic exosome.

PEG conjugated phospholipids are well known to those of skill in the art. Illustrative PEG conjugated phospholipids include, but are not limited to phosphoethanolamine-PEG lipids such as dipalmitoyl-sn-glycero-3-phospho(ethylene glycol) (DPPEG1), dimyristoyl-sn-glycero-3-phosphoethanolamine-N-[methoxy(polyethylene glycol)-350 (DMPEG350), distearoyl-sn-glycero-3-phosphoethanolamine-N-[methoxy(polyethylene glycol)-350] (DTPEG350), dimyristoyl-sn-glycero-3-phosphoethanolamine-N-[methoxy(polyethylene glycol)-550] (DMPEG550), and dimyristoyl-sn-glycero-3-phosphoethanolamine-N-[methoxy(polyethylene glycol)-1000] (DMPEG1000), and the like.

In certain embodiments additionally, or alternatively, the synthetic exosomes are pegylated by incorporation of a PEG functionalized cholesterol and/or a PEG functionalized cholesterol derivative.

The foregoing pegylation options are illustrative and non-limiting. Using the teaching provided herein numerous other pegylation schemes for formation of pegylated synthetic exosomes will be available to those of skill in the art.

### Synthetic Exosome (SE) Synthesis Using Microfluidic Flow Reactors.

In one illustrative embodiment a microfluidic reactor is used to synthesize the synthetic exosomes. In certain embodiments the microfluidic reactors uses three pumps to flow three fluids into the microfluidic chip. In certain embodiments, two of the streams are water and the other stream is isopropyl alcohol (IPA) that may contain components of the liposome.

In certain embodiments, the surfactant is provided in the water stream.

In certain embodiments the aqueous stream flow rate can range from 0.5 mL/min-10 mL/min, depending on the particle size desire and contains any biologics if needed. Each one of the flow rates can be manipulated individually.

In certain embodiments the organic stream flow rate can range from 0.05 mL/min-5 mL/min, depending on the particle size desire and contains the lipids mixture and any hydrophobic small molecule if needed.

In certain embodiments the microflow fluidic flow reactor utilizes one or more organic stream that contain components of the lipid bilayer (e.g., cholesterol, phospholipid, surfactant), and one or more aqueous (e.g., water) streams. In certain embodiments the organic stream comprises an alcohol (e.g., isopropyl alcohol) in addition to the components of the lipid bilayer.

In various embodiments the therapeutic moiety (e.g., base editor and/or guide RNA) is provided in the stream that is most likely to suspend or dissolve the moiety. Thus, for example, in typical embodiments, a hydrophobic therapeutic moiety is provided in the organic stream, while hydrophilic moieties are provided in the aqueous stream. Hydrophilic moieties such as peptides, enzymes, proteins and antibodies, nucleotides, DNA, and the like can be provided in the aqueous stream. It will be recognized that, in certain embodiments, two, three, or four different therapeutic moieties can be loaded into each synthetic exosome.

In certain illustrative, but non-limiting embodiments, the organic stream lipid mixture concentration ranges from about 5 mM to about 20 mM and any hydrophobic cargo will range from 0.05 mM up to about 2 mM depending on the cargo solubility. In certain illustrative, but non-limiting embodiments, the aqueous stream contains a hydrophilic molecule its concentration ranges from about 0.01 mg/mL to about 5 mg/mL depending on its solubility.

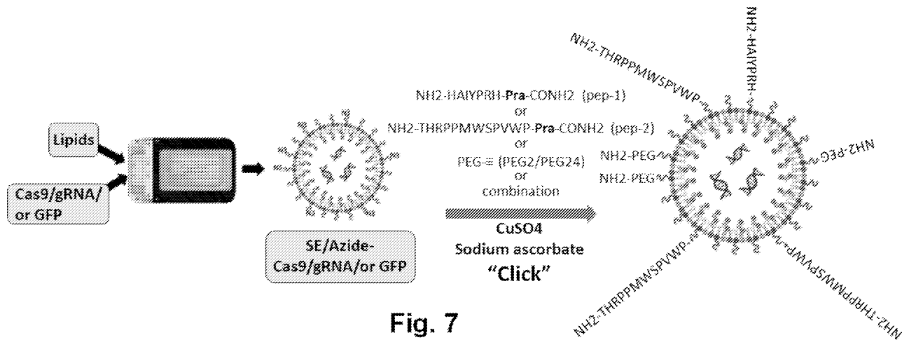

For the synthetic exosome synthesis, we have used two commercially available reactors: a 26 μL and the 1000 μL reactors but other reactors are also possible. The design of two illustrative reactors is shown in PCT Application No: PCT/US2021/026049, filed on Apr. 6, 2021 (see, e.g., FIG. 7) which PCT Application is incorporated herein by reference for the microfluid reactors described therein.

In order to improve the SE synthesis, we can use custom made reactors, their design is shown in FIG. 8 of PCT Application No: PCT/US2021/026049. As shown in the illustrative embodiment shown in FIG. 8 of PCT Application No: PCT/US2021/026049 the microfluidic flow reactor comprises a central channel with two or more branch channels feeding the central channel and thereby forming a mixing junction, where the diameter of said central channel and branch channels and the angle provided between said central channel and branch channels are selected to maintain a backpressure of less than about 100 psi. The design is based on minimizing turbulence in the junction while maintaining backpressure of less than 100 psi, the angle of impact in the mixing junction allows us to minimize turbulence while the channel width and height allow us to control the pressure.

In certain embodiments the width(s) of said central channel and/or branch channels independently range from about 0.5 μm or from about 1 μm, or from about 10 μm, or from about 20 μm, or from about 30 μm up to about 100 μm, or up to about 80 μm, or up to about 60 μm, or up to about 50 μm, or up to about 40 μm. In certain embodiments the height(s) (depth(s)) of the central channel and/or branch channels independently range from about 0.5 μm or from about 1 μm, or from about 10 μm, or from about 20 μm, or from about 30 μm up to about 100 μm, or up to about 80 μm, or up to about 60 μm, or up to about 50 μm, or up to about 40 μm. In certain embodiments the angle (a) between the central channel and said lateral channels ranges from about 10°, or from about 15°, or from about 20°, or from about 25° up to about 90°, or up to about 80°, or up to about 70°, or up to about 60°, or up to about 50°. In certain embodiments the reactor comprises one or more pumps where said pumps provide a fluid pressure ranging from about 1 bar to about 31 bar.

One embodiment of the microfluidic reactor system is shown in FIG. 9 of PCT Application No: PCT/US2021/026049 and its elements are highlighted therein.

### Functionalization of the Lipids in Series

Synthetic exosome synthesis using the microfluidic reactor also enables us to use functionalized lipids such as the one listed in Table 4 below to tag different ligands to the synthetic exosome in series. This can be achieved with the use of reactors connected in series or with the design of the new reactor shown in FIG. 10 in PCT Application No: PCT/US2021/026049. As shown in the illustrative embodiment shown in FIG. 10 in PCT Application No: PCT/US2021/026049 the microfluidic flow reactor comprises a central channel with two or more branch channels feeding the central channel and thereby forming a first mixing junction, and a second set of branch channels feeding the central channel and thereby forming a second mixing junction. It will be recognized that the reactor can contain additional mixing junctions. Thus, reactors comprising 2, 3, 4, 5, 6 or more mixing junctions are contemplated. In certain embodiments the diameter of the central channel and branch channels and the angle provided between the central channel and branch channels are selected to maintain a backpressure of less than about 100 psi. Again, the design is based on minimizing turbulence in the junction while maintaining backpressure of less than 100 psi, the angle of impact in the mixing junction allows us to minimize turbulence while the channel width and height allow us to control the pressure.

For the synthesis, after the SE are formed in the first mixing junction, then the ligand can react with the SE in the second mixing junction. Additionally, in certain embodiments more tagged lipids can be reacted with the forming SE in additional mixing junctions. All of the reactions can be independently tuned.

In certain illustrative, but non-limiting embodiments the “tagged” synthesis facilitates the use of specific proteins ligands that can include, but are not limited to, ligands that will have a receptor in the BBB. Illustrative ligands include, but are not limited to insulin, transferrin, low density lipoprotein receptor-related protein 1, or any other ligand such as an amino acid.

In certain embodiments one of skill can use the lipid NHS ester and then functionalize it with various ligands such as peptide, proteins, amino acids, etc.

TABLE 4
Illustrated functionalized lipids can be purchased
or prepared by “tagged” synthesis.
Functionalized Lipid

Dioleoyl-sn-glycero-3-phosphocholine (N-aminoethyl)
Dioleoyl or dipalmitoyl-sn-glycero-3-phosphoethanolamine-N-[4-(p-
maleimidomethyl)cyclohexane-carboxamide]
Dipalmitoyl-sn-glycero-3-phosphoethanolamine-N-[4-(p-
maleimidophenyl)butyramide]
Dipalmitoyl-sn-Glycero-3-Phosphothioethanol
Dipalmitoyl-sn-glycero-3-phosphoethanolamine-N-(succinyl)
Distearoyl-sn-glycero-3-phosphocholine (N-propynyl)
Dipalmitoyl-sn-glycero-3-phosphoethanolamine-N-dibenzocyclooctyl
Distearoyl-sn-glycero-3-phosphocholine (N-azidoethyl)
Dimyristoyl-sn-glycero-3-phosphoethanolamine-N-[methoxy(polyethylene
glycol)-350]
Dimyristoyl-sn-glycero-3-phosphoethanolamine-N-[methoxy(polyethylene
glycol)-550]
Dimyristoyl-sn-glycero-3-phosphoethanolamine-N-[methoxy(polyethylene
glycol)-750]
Dimyristoyl-sn-glycero-3-phosphoethanolamine-N-[methoxy(polyethylene
glycol)-1000]
Dimyristoyl-sn-glycero-3-phosphoethanolamine-N-[methoxy(polyethylene
glycol)-2000]
Distearoyl-sn-glycero-3-phosphoethanolamine-N-[carboxy(polyethylene
glycol)-2000]
Distearoyl-sn-glycero-3-phosphoethanolamine-N-[amino(polyethylene
glycol)-2000]
Distearoyl-sn-glycero-3-phosphoethanolamine-N-[succinyl(polyethylene
glycol)-2000]
Distearoyl-sn-glycero-3-phosphoethanolamine-N-[maleimide(polyethylene
glycol)-2000]
Distearoyl-sn-glycero-3-phosphoethanolamine-N-[biotinyl(polyethylene
glycol)-2000]
Distearoyl-sn-glycero-3-phosphoethanolamine-N-[carboxy(polyethylene
glycol)-2000, NHS ester]
Dipalmitoyl-sn-glycero-3-phosphoethanolamine-N-[azido(polyethylene
glycol)-2000]
Distearoyl-sn-glycero-3-phosphoethanolamine-N-[azido(polyethylene
glycol)-2000]
Dioleoyl-sn-glycero-3-phosphoethanolamine-N-[azido(polyethylene
glycol)-2000]
Dioleoyl-sn-glycero-3-phosphoethanolamine-N-[azido(polyethylene
glycol)-2000]
Dioleoyl-sn-glycero-3-phosphoethanolamine-N-[amino(polyethylene
glycol)-2000]
Dioleoyl-sn-glycero-3-phosphoethanolamine-N-[carboxy(polyethylene
glycol)-2000]
Dimyristoyl-sn-glycero-3-phosphoethanolamine-N-[methoxy(polyethylene
glycol)-3000]
Dimyristoyl-sn-glycero-3-phosphoethanolamine-N-[methoxy(polyethylene
glycol)-5000]
Distearoyl-sn-glycero-3-phosphoethanolamine-N-[amino(polyethylene
glycol)-5000]
16:0 Azidoethyl sphingomelin
1,2-distearoyl-sn-glycero-3-phosphocholine (N-azidoethyl)
(2S,3R,E)-2-amino-14-azidotetradec-4-ene-1,3-diol
1,2-distearoyl-sn-glycero-3-phosphoethanolamine-N- [azido
(polyethylene glycol)-2000]

The foregoing synthetic exosome (SE) formulations and synthesis procedures are illustrative and non-limiting. Using the teaching provide herein numerous alternative SE formulations and synthesis protocols for preparation of synthetic exosomes containing base editors will be available to one of ordinary skill in the art.

### Pharmaceutical Formulations.

In various embodiments pharmaceutical formulations contemplated herein contain synthetic exosomes as described herein and a pharmaceutically acceptable carrier. The term “carrier” typically refers to an inert substance used as a diluent or vehicle for the pharmaceutical formulation. The term can also encompass a typically inert substance that imparts cohesive qualities to the composition. Typically, the physiologically acceptable carriers are present in liquid form. Examples of liquid carriers include, but not limited to, physiological saline, phosphate buffer, normal buffered saline (135-150 mM NaCl), water, buffered water, 0.4% saline, 0.3% glycine, 0.3M sucrose (and other carbohydrates), glycoproteins to provide enhanced stability (e.g., albumin, lipoprotein, globulin, etc.) and the like. Since physiologically acceptable carriers are determined in part by the particular composition being administered as well as by the particular method used to administer the composition, there are a wide variety of suitable formulations of pharmaceutical compositions of the present invention (see, e.g., Remington's Pharmaceutical Sciences, Maak Publishing Company, Philadelphia, Pa., 17th ed. (1985)).

In various embodiments the pharmaceutical formulations can be sterilized by conventional, well-known sterilization techniques or may be produced under sterile conditions. Aqueous solutions can be packaged for use or filtered under aseptic conditions and lyophilized, the lyophilized preparation being combined with a sterile aqueous solution prior to administration. In certain embodiments the compositions can contain pharmaceutically acceptable auxiliary substances as required to approximate physiological conditions, such as pH adjusting and buffering agents, tonicity adjusting agents, wetting agents and the like, e.g., sodium acetate, sodium lactate, sodium chloride, potassium chloride, calcium chloride, sorbitan monolaurate and triethanolamine oleate. Sugars can also be included for stabilizing the compositions, such as a stabilizer for lyophilized compositions.

Pharmaceutical compositions suitable for parenteral administration, such as, for example, by intraarticular, intravenous, intramuscular, intratumoral, intradermal, intraperitoneal and subcutaneous routes, can include aqueous and non-aqueous, isotonic sterile injection solutions. In certain embodiments the injection solutions can contain antioxidants, buffers, bacteriostats and solutes that render the formulation isotonic with the blood of the intended recipient, and aqueous and non-aqueous sterile suspensions that can include suspending agents, solubilizers, thickening agents, stabilizers and preservatives. Injection solutions and suspensions can also be prepared from sterile powders, such as lyophilized synthetic exosomes. In certain embodiments the compositions can be administered, for example, by intravenous infusion, intraperitoneally, intravesically or intrathecally. In various embodiments parenteral administration and intravenous administration are also contemplated. The formulations of liposome compositions can be presented in unit-dose or multi-dose sealed containers, such as ampoules and vials.

In certain embodiments the pharmaceutical compositions are formulated for systemic administration as an injectable.

In certain embodiments the pharmaceutical compositions are formulated for administration as an aerosol, e.g., for oral and/or nasal inhalation.

In certain embodiments the pharmaceutical compositions are formulated for topical deliver, intradermal delivery, subdermal delivery and/or transdermal delivery.

In certain embodiments the pharmaceutical compositions are formulate for application to oral mucosa, vaginal mucosa, and/or rectal mucosa.

In certain embodiments the pharmaceutical composition is in unit dosage form. In such form, the composition is subdivided into unit doses containing appropriate quantities of the active component (synthetic exosome). The unit dosage form can be a packaged composition, the package containing discrete quantities of the pharmaceutical composition. The composition can, if desired, also contain other compatible therapeutic agents.

In certain embodiments the synthetic exosomes described herein can be delivered through the skin using conventional transdermal drug delivery systems, i.e., transdermal “patches” wherein the synthetic exosomes or formulations thereof) are typically contained within a laminated structure that serves as a drug delivery device to be affixed to the skin. In such a structure, the synthetic exosomes and/or formulations thereof are typically contained in a layer, or “reservoir,” underlying an upper backing layer. It will be appreciated that the term “reservoir” in this context refers to a quantity of synthetic exosomes, and/or formulations thereof that is ultimately available for delivery to the surface of the skin. Thus, for example, the “reservoir” may include the active ingredient(s) in an adhesive on a backing layer of the patch, or in any of a variety of different matrix formulations known to those of skill in the art. The patch may contain a single reservoir, or it may contain multiple reservoirs.

In one illustrative embodiment, the reservoir comprises a polymeric matrix of a pharmaceutically acceptable contact adhesive material that serves to affix the system to the skin during drug delivery. Examples of suitable skin contact adhesive materials include, but are not limited to, polyethylenes, polysiloxanes, polyisobutylenes, polyacrylates polyurethanes, and the like. Alternatively, the synthetic exosome and/or synthetic exosomes formulation reservoir and skin contact adhesive are present as separate and distinct layers, with the adhesive underlying the reservoir which, in this case, may be either a polymeric matrix as described above, or it may be a liquid or hydrogel reservoir, or may take some other form. The backing layer in these laminates, which serves as the upper surface of the device, preferably functions as a primary structural element of the “patch” and provides the device with much of its flexibility. The material selected for the backing layer is preferably substantially impermeable to the synthetic exosomes and/or formulations thereof) and any other materials that are present.

Alternatively, other pharmaceutical delivery systems can be employed. For example, emulsions, and microemulsions/nanoemulsions are well known examples of delivery vehicles that may be used to protect and deliver pharmaceutically active compounds. Certain organic solvents such as dimethylsulfoxide also can be employed, although usually at the cost of greater toxicity.

Methods of Use—Treatment and/or Prophylaxis of Neurological Pathologies.

AS noted above, it is believed that synthetic exosomes containing a base editor that targets an ApoE4 gene as described herein can be used in the treatment and/or prophylaxis of a number of pathologies implicating ApoE4 and/or ApoE4 associated pathways. In certain embodiments such a pathologies include but are not limited to Alzheimer's disease (AD), frontotemporal dementia, cerebral amyloid angiopathy (CAA), dementia with Lewy bodies (DLB), tauopathy, cerebrovascular disease, multiple sclerosis, vascular dementia, Parkinson's disease, and treatment after traumatic brain injury and/or hemorrhage.

Alzheimer's disease (AD) is a progressive neurodegenerative disorder characterized clinically by memory and cognitive dysfunction. The disease is generally classified into two types: sporadic AD (SAD) and familial AD (FAD). Three genes lead to familial AD (FAD)—amyloid precursor protein (APP), presenilin 1 (PS1), and presenilin (PS2)—while the &4 allele of the apolipoprotein E gene has been identified as the major risk factor for sporadic AD (SAD) The neuropathology of AD is characterized by two types of lesions, extracellular senile plaques and intracellular neurofibrillary tangles (NFTs), that are composed of, respectively, β-amyloid (Aβ), a cleavage product of APP, 4 and aberrantly phosphorylated tau, a microtubule-associated protein.

Research has established that most neurodegenerative diseases including Alzheimer's, Lewy Body and other dementias, Parkinson's and prion diseases develop and progress along similar paths. In each disease, a particular protein undergoes a change in its shape from a soluble, physiologically functional protein to a protein that has lost the ability to perform its required tasks in the brain, starting off a chain reaction of binding to each other.

Accordingly, in certain embodiments prophylactic and/or treatment methods are provided that involve administration to a subject in need thereof (e.g., at risk for or having the neurodegenerative disease) a synthetic exosome as described herein containing a CRISPR constructs for editing a gene encoding ApoE4 as described herein.

### Kits.

In certain embodiments kits for the delivery of a therapeutic moiety (e.g., a gene editor that targets and edits a gene encoding ApoE4) to the brain are provided. Typically, such kit will comprise a container containing synthetic exosomes (SEs), containing the gene editor and associated guide RNA. In certain embodiments the synthetic exosomes can be provided in a unit dosage formulation (e.g., vial, tablet, caplet, patch, etc.) and/or may be optionally combined with one or more pharmaceutically acceptable excipients.

In addition, in certain embodiments, the kits optionally include labeling and/or instructional materials providing directions (i.e., protocols) for the use of the synthetic exosomes described herein. Thus, for example, the kit may contain directions for the use of the synthetic exosomes comprising a therapeutic moiety in the treatment of dementia, mild cognitive impairment, Alzheimer's disease, Amyotrophic lateral sclerosis (ALS), Parkinson's disease, cerebral amyloid angiopathy, and the like as well as for Traumatic brain injury (TBI) and Stroke therapy or for treatment of a brain cancer. In various embodiments the instructional materials may also, optionally, teach preferred dosages/therapeutic regiment, counter indications and the like.

While the instructional materials typically comprise written or printed materials, they are not limited to such. Any medium capable of storing such instructions and communicating them to an end user is contemplated by this invention. Such media include but are not limited to electronic storage media (e.g., magnetic discs, tapes, cartridges, chips), optical media (e.g., CD ROM), and the like. Such media may include addresses to internet sites that provide such instructional materials.

### EXAMPLES

The following examples are offered to illustrate, but not to limit the claimed invention.

### Example 1

Encapsulation of Cas9 and/or IDUA

### Microfluidic Reactor Synthesis of SEs.

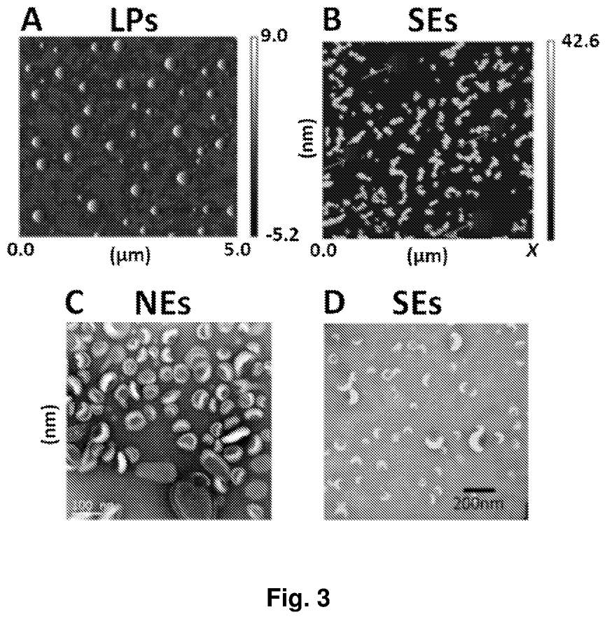

SEs with encapsulated cargo form when the lipid and aqueous (typically carrying the cargo) streams mix in the reactor [18]. The lipids and other non-cargo components are Generally Regarded as Safe (GRAS) materials. The microfluidic reactor flow rates can be finely controlled to yield SEs with specific size (60<φ<500 nm), zeta potential (−50<ξ<50), and deformability. Once optimized, microfluidic synthesis of SEs is readily scalable to obtain larger amounts and allows good batch-to-batch reproducibility. After synthesis, SEs encapsulating a therapeutic are characterized, dialyzed, and lyophilized before storage (see, e.g., FIG. 3). Lyophilized SEs can be stored for months before resuspension in vehicle and use.

The morphological characteristics of SEs as compared to conventional liposomes (LPs) and natural exosomes (NEs) are shown in FIG. 4. Atomic Force Microscopy (AFM) of LPs and SEs reveals SEs (FIG. 4, panel B) but not LPs (FIG. 4, panel A) are deformable. Transmission Electron Microscopy (TEM), used to determine SE size, shows the morphological similarities between NEs and our SEs, including size and a crescent-like shape (FIG. 4, panels C and D).

### Successful SE Delivery of Proteins to the Brain.

To demonstrate SE-encapsulated proteins can be delivered to brain, we first synthesized SE-soluble amyloid precursor protein alpha (SE-sAPPα; 678 amino acids); the characteristics are shown in FIG. 5, panel A. sAPPα is an inhibitor of the enzyme BACE1 [41], which cleaves of full-length APP resulting in production of sAPPβ and ultimately amyloid-β (Aβ). Two days after delivery, decreases in sAPPβ and Aβ were greater with SE-sAPPα than free sAPPα in CHO-7W cells (FIG. 5, panel B).

We then compared SE-sAPPα, free sAPPα and vehicle in human APP-expressing (huAPP) mice after tail vein injection. Levels of human sAPPα in brain were highest at 1 hr post-delivery of SE(ζ−)-sAPPα (FIG. 5, panel D), at a physiologically relevant level of 12 nM and was significantly higher than free sAPPα, which did not enter the brain. In huAPP mouse brain, sAPPβ showed a trend to be lower with SE-sAPPα than either free sAPPα or vehicle (FIG. 5, panel E).

A second protein, the enzyme iduronidase (IDUA), was also encapsulated in SEs. The characteristics of negatively and positively charged SE-IDUA are shown in FIG. 5, panel F. After IV delivery of SE-IDUA to IDUA knockout (−/−) mice, IDUA brain levels were significantly higher with SE(ζ−)-IDUA than SE(ζ+)-IDUA or free IDUA (FIG. 5, panel G), highlighting the role of SE charge in brain penetrance.

A third protein, the enzyme acid sphingomylienase (ASM), was also encapsulated in SEs. After IV delivery of SE-ASM to wild type mice, ASM brain activity were significantly higher with SE(ζ+)-ASM (FIG. 5 panel H), highlighting the role of SE charge in brain penetrance.

Finally, we encapsulated Cas9 with a his-tag (SE-Cas9-his), delivered 10 μg to non-transgenic mice by tail vein injection, and collected brain tissue 1 hr post-dosing for detection of Cas9-his by ELISA. As shown in FIG. 5, panel I, brain levels of Cas9-his were significantly higher with SE(−)-Cas9-his than free Cas9-his.

CRISPR gRNA Design and Testing.

We designed candidate gRNAs using the ChopChop web tool [42], intended for a standard homology-directed repair (HDR) CRISPR approach to E4 to E3 editing, and provide proof-of-principle to be applied to the base editing gRNA that would be used in this proposal. Briefly, 6 candidates were designed, synthesized, complexed with Cas9 protein, and electroporated into N2a-E4 cells using different Cas9:gRNA ratios. PCR primers amplified the 305 bp region with the target E4 site in the middle, the products were Sanger sequenced, and subjected to analysis by Inference of CRISPR Edits (ICE) for INDEL frequency. We found that the Cas9:gRNA ratio of 1.5:1 resulted in the highest INDEL frequency of 52%.

### Example 2

### Encapsulation of Gene Editing Cas9

### Specific Aims.

The major genetic risk factor for sporadic Alzheimer's disease (AD) is the &4 allele of the apolipoprotein gene (ApoE4; E4). E4 expression influences the development of both tau and amyloid-beta (Aβ) pathology and their spread throughout the brain [4, 6, 7, 12, 13]. The risk conferred by E4 may potentially be decreased by converting E4 to E3. One suggested approach to conversion of E4 to E3 is by use of protein structure-modifying drugs [14]. An alternative approach, described in this proposal, is to edit the E4 gene sequence to E3 in the brain by use of CRISPR base editing (BE) technology. CRISPR technology is being used to edit genes involved in a wide range of diseases, including AD [15], and clinical trials of gene editing for cancer and diseases of the eye are underway [16, 17]. For AD, a major impediment to genetic editing in the brain is the blood-brain-barrier (BBB). To address this challenge, we plan to deliver the CRISPR components that edit E4 to E3 in synthetic exosomes (SEs), a type of deformable nanovesicle the size of a natural exosome that can encapsulate biomolecules during microfluidic reactor synthesis, a method well-established in our lab [18]. Our preliminary data show that SEs can cross the BBB and deliver proteins to the brain tissue of mice. We have begun testing CRISPR guide RNA (gRNA) in vitro and achieved the first step in CRISPR editing—the creation of insertion-deletion (INDEL) mutations in the ApoE4 gene at the site of that distinguishes E4 from E3, a single nucleotide in the codon for amino acid 112. In this example we optimize SEs encapsulating BE CRISPR components nCas9 (Cas9 nickase D10A mutant fused to cytidine deaminase & uracil glycosylase inhibitor) and gRNA (SE-CRISPR) for in vivo delivery to the brain of E4 targeted-replacement (E4 TR) mice and assess the ability of SE-CRISPR to edit E4 to E3.

### Design and Optimization of SE Synthesis and SE-Delivered CRISPR Components.

We are continuing our design and testing of CRISPR components for editing E4 to E3 using murine neuroblastoma N2a cells that stably express E4 (N2a-E4).

### BE CRISPR Design, Testing, and Optimization.

We have designed a candidate BE gRNA and identified a Cas9n PAM-specificity version, an important feasibility step. The BE gRNA activity is evaluated by electroporation of BE and gRNA at varying ratios into N2a-E4 cells and determining the E4:E3 ratio using quantitative Sanger sequencing. Any off-target editing, if seen, will inform BE gRNA design, for which other options are available.

### Optimization of Microfluidic Reactor SE-CRISPR Synthesis.

Microfluidic reactor synthesis of SE-CRISPR is optimized for SE size, charge, and encapsulation efficiency by measuring average encapsulated Cas9n per SE. SEs encapsulating Cas9-green fluorescent protein (Cas9-GFP) are synthesized with the azide phospholipids needed for “click chemistry”-based surface modification with transferrin (Tf) peptides and/or polyethylene glycol (PEG), which are anticipated to increase brain permeability [19, 20]. Any increases in brain permeability are assessed as described below.

### SE-CRISPR Editing of N2a-E4 Cells.

N2a-E4 cells (available in the lab) are incubated with SE-CRISPR with an optimal BE:gRNA ratio for 3 days and the E3:E4 ratio determined.

### Delivery of SE-CRISPR in E4 TR Mice and Assessment of E4:E3 Gene Editing in the Brain.

Optimized SE-CRISPR are delivered to E4 TR mice for assessment of the efficiency of genetically editing E4 to E3.

### Pharmacokinetic Analysis of Unmodified & Surface-Modified SEs.

To determine the SE design providing the greatest brain permeability, PK analysis of unmodified and surface-modified SE-Cas9-GFP is performed and brain levels of GFP post-intravenous (IV) injection are determined by total brain GFP fluorescence, GFP+ cell count by flow cytometry, and fluorescence imaging of brain tissue sections.

### Delivery of SE-CRISPR to E4 TR Mouse Brain and Editing of E4 to E3.

SE-CRISPR optimized for both CRISPR base editing components and SE design are delivered by IV tail vein injection to E4 TR mice [21]. Mice are euthanized at 7 days and 1 and 6 months with an n=4 for each time point and an SE-Empty (no CRISPR) control group, also n=4. Brain tissue is analyzed by region (frontal and parietal cortex, hippocampus, thalamus and striatum) for E4:E3 editing efficiency. Day 7 analyses will show initial E4 to E3 editing and the later time points stability of editing as cells turnover, proliferate and differentiate. At 6 months, editing efficiency in various cell types such as neurons, astrocytes, microglia, and endothelia is determined. E4 and E3 protein levels by region are assessed by targeted mass spectroscopy and correlated to the E3:E4 gene editing ratio.

### Editing Effects Outside of the Brain.

Other organs and tissues, such as the liver, germ line cells and blood, are collected for analysis including E4 to E3 gene editing and levels of E4 and E3 protein.

### Methods.

### PAM Site Identification and Bystander Editing Analysis.

The many advantages of base editing include increased efficiency (>20% to 80%) and reduced INDELs (<1%); its disadvantages are precise PAM site spacing and bystander editing [36, 37, 39]. Base editing requires precise positioning of the Cas9-linked deaminase by gRNA over the target DNA site (see FIG. 1) which abuts a nearby PAM sequence. We analyzed the E4 sequence and identified a candidate PAM (FIG. 6). The E4 sequence of the protospacer sequence numbering relative to PAM (TG) includes the deaminase peak activity window, which is from base 4 to 8, but peripheral deaminase activity can extend from base 2 to 11. Komor et al. (2016) Nature, 533: 420-424, demonstrated that the E4 C in codon 112 can be deaminated in vitro. The deaminase activity is a 5 bp window, so other bystander Cs can be deaminated and would be evaluated for mutation effects. All possible deaminations of bystander Cs, result in silent mutations; the C at position 4 in codon 110 (GAC to GAT) code for asparagine; and both Cs at 8 and 10 (outside peak activity window; TGC to TGT) code for cysteine. Other potential C bystanders at 10 and 13, outside peak or peripheral activity windows, encode arginine and glycine, respectively.

In certain embodiments the gRNA is the same 20 nt sequence as the protospacer sequence abutting the PAM. This gRNA would be synthesized (Synthego) with an 80 nt tracrRNA that specifically binds Cas9 protein (“sgRNA” in FIG. 1). Importantly, the sequence of this region of E4 will be verified by Sanger sequencing in both the models used: the N2a-E4 cells and E4 TR mice.

The BE tool Cas9 is a fusion of 3 proteins (FIG. 1), with BE4 being the latest generation (Caso & Davies (2021) Laboratory animals, PMID: 33596731; Vasquez et al. (2020) Curr. Protocol. Mol. Biol. 133: e129; Antoniou et al. (2021) Front. Genome Editing, 3: 618406. doi: 10.3389). The Cas9 PAM recognition activity will be for the TG PAM site designed above, specifically SpCas9n-NG (Nishimasu et al. (2018) Science (New York, N.Y.), 361: 1259-1262). We use a Cas9 bacterial expression vector to produce bulk protein, and this will be combined with gRNA in a BE:gRNA complex for encapsulation in SEs.

To optimize the BE-gRNA, its editing activity is predicted in silico using BE-Hive [45, 46] and validated in vitro by electroporation of the BE:gRNA complex into N2a-E4 cells using different ratios of components; the negative control will not have gRNA. Genomic DNA will be PCR amplified, Sanger sequenced with high quality results, and base-editing efficiency (on target and bystander) quantified using EditR [47]. Sanger/EditR is valid for >10% efficiency; for <10%, NGS (using Amplicon-EZ (Genewiz.com)) at 50K reads (using the fewest possible PCR cycles) and analyzed using CRISPResso2 [48]. BE:gRNA combination optimization balances on-target efficiency with undesired INDELs around the E4 site and off-target editing. INDELs will also be quantified from Sanger (sensitivity limit >5%) using ICE [43]. The 10 top most probable sites for gRNA-dependent off-target effects will be predicted using Cas-OFFinder [49], then each site will undergo PCR amplification followed by Sanger sequencing and ICE analysis.

We expect at least moderate efficiency (>20%) with infrequent off-target editing. Once the BE and gRNA are validated, large-scale synthesis of gRNA (Synthego) and the Cas9/BE (using bacterial expression bulk production at UCLA core) will generate sufficient material for SE encapsulation for in vitro and in vivo studies.

### Optimization of Microfluidic SE-CRISPR Synthesis.

BE/gRNA will be encapsulated in SEs during synthesis. Encapsulation efficiency (ee) will be determined by counting the SE particles stained with the bright fluorescent membrane dye DiO by Nano Flow using our AttuneNxt flow cytometer, quantifying total Cas9 by Cas9 ELISA, and calculating the average Cas9/SE particle. To determine the maximum encapsulation efficiency, we will synthesize SE-Cas9 using three different concentrations of Cas9 (1, 10 and 20 uM Cas9), and then use the optimized concentration of Cas9 for SE-BE(Cas9)/gRNA synthesis. BE/gRNA will enter the reactor in the aqueous stream at 25° C. with all solutions and components RNase-free; it is expected to be stable, but we will test the editing activity of the SE-BE/gRNA preparation on N2a cells in vitro.

### Surface Modification of SEs

We will also synthesize and characterize SE-Cas9-GFP with the azide phospholipids needed for click-chemistry-based modification of SE surface with Tf peptide motifs and PEG. Tf peptides have been shown to target the Tf receptor at the BBB and facilitate brain entry [19, 50] and PEG [20, 51-55] to increase circulating half-life. The goal is to compare the surface modified SE to unmodified SE's for increased BBB permeability in PK analysis followed by use for proof-of-concept delivery of CRISPR in the mouse model in Aim 2.

Method for Click Chemistry Labelling of SEs with PEG and Tf.

As shown in FIG. 7, at room temperature and in the presence of CuSO4, phosphate-terminal azides displayed on the surface of SEs synthesized using lipid azides (Avanti Polar Lipids, AL; 870131P, 870130P, and 870126P) undergo a cycloaddition reaction to form covalent bonds with the propargyl (HC≡C—CH2-) alkyne (Pra) group of either PEG (BroadPharm, CA; BP-22519, BP-21683) or the Tf peptides (Bachem, CA; custom). The yield of the click reaction is very high [56, 57]. In FIG. 7, Tf peptides with either 7 or 12 amino acids or 2 PEG moieties (PEG2) or 24 moieties (PEG24) are shown.

### SE-CRISPR Editing of E4 to E3 in N2a-E4 Cells.

N2a-E4 cells will be incubated with SE-CRISPR and the E3:E4 gene sequence ratio determined by Sanger sequencing. The SE-CRISPR concentration will be varied between 5-50 pmoles Cas9 with the goal of editing >20%, possibly as high as 75%, based on current literature.

### Alternative Strategies.

If the editing efficiency is not high, we will test other modular BE:gRNA combinations that have an expanded editing window (positions 3112) combined with TGG PAM recognition which positions the target E4 C at position 11. C methylation at the target CG site can be deaminated with hA3A-BE3. GC context inhibition can be overcome with evoCDA1-BE4max and new generation BE variants are constantly being generated [39].

### Delivery of SE-CRISPR in E4 TR Mice and Assessment of E4:E3 Gene Editing in the Brain.

We will assess the brain-permeability of surface-modified SEs to select the best SE type for use in proof-of-concept studies of SE-CRISPR delivery and E4 to E3 editing in E4 TR mouse brain in vivo.

### Methods

### Pharmacokinetic Analysis of Unmodified and Surface Modified-SEs for Brain Permeability.

Brain levels of modified and unmodified SEs (Tf, PEG and Tf+PEG) encapsulating Cas9-Green Fluorescent Protein fusion (Cas9-GFP; synthesized by the UCLA Protein Expression Core) and an SE-empty control will be compared by IV tail vein injection of E4 TR mice (n=4 per group). Three SE-Cas9-GFP doses for each type of SE will be tested to establish the maximum dose. Mice will be anesthetized, perfused with saline and euthanized 2, 4 and 12 hours post-injection. Blood and brain (frontal and parietal cortex, hippocampus, thalamus, and striatum) from a hemi-brain will be collected. The other hemi-brain will be fixed in 2% PFA for fluorescence imaging of sections. GFP levels will be determined by the GFP signal on a plate reader and by flow cytometry of GFP+ cells from cell suspensions, and by cellular GFP fluorescence detection in tissue sections. Detection of intact DE-Cas9-GFP puncta in brain may require loading SEs with ultrabright mGreen Lantern GFP.

### Assessment of Immune Markers.

To determine if SEs, modified or unmodified, elicit an immune reaction, in all animals in Aim 2 we will measure blood levels of cytokines and other immune markers (histamine, IL-6, IP-10, TNFα, and C3) by EIA and ELISA [50]. The presence of hemolysis, by PEG, will also be determined [50].

### SE-CRISPR Editing of E4 to E3 in E4 TR Mice.

Using the best SE type and CRISPR components and ratios as determined above, we will inject E4 TR mice and evaluate E4 to E3 editing over a time course of 7 days to 6 months. Synthesis of SE-CRISPR will be scaled up to produce sufficient amounts for these studies. As noted above, SEs can be synthesized then lyophilized and stored before use. There will be 6 groups, SE-CRISPR and SE-Empty and 3 time points for euthanasia (7 days; 1 and 6 months) with n=4 per group (2 female and 2 male) E4 TR mice. The maximum dose as determined above will be injected via the tail vein and mice will be euthanized by pentobarbital over-anesthesia, blood collection and transcardial saline perfusion before collection of a hemi-brain for dissection into regions and a hemi-brain for fixation and sectioning, as described above. Liver, testes, and ovaries will also be collected for future analysis.

### E4 to E3 Editing Efficiency In Vivo.

The level of E4 to E3 editing will be quantified by Sanger sequencing (non-rare events or >5% editing). To detect rare E3 editing events, we will use NGS (Amplicon-EZ, Genewhiz) on PCR-amplified E4 region, at a depth of 50K reads. To detect very rare E4-E3 editing events, we will use high-fidelity redundant sequencing of the E4-region amplicon with bar-coded PCR primers and linear followed by exponential amplification. Off-target effects such as INDELs near E4 and gRNA-dependent events in the E4-TR mouse brain will be evaluated as described above. gRNA-independent effects will be assessed by whole-genome or whole-exome sequencing of brain tissue from one 6-month old mouse. Mice euthanized at Day 7 will represent short-term editing effects and those euthanized at 1 and 6 months represent long-term editing stability that will likely be affected by brain cell turnover, proliferation, and differentiation effects.

In the 6 month time point animals, to determine the brain cell types expressing E3, brain regions will be converted to single cell suspension, fixed, and major brain cell types will be purified using the Neural Tissue Dissociation kit (Miltenyi MACS). Briefly, suspensions are depleted of all non-neuronal cells which leaves behind neurons, then non-neuronal cells are sequentially exposed to these cell type-specific antibody-coupled MACS magnetic beads: astrocyte: anti-ACSA-2; microglia/macrophage: anti-CD11b (which are further purified with microglia-specific anti-TMEM119 [59, 60]; endothelia: anti-CD31. Cell populations will be counted and sequenced by Sanger and NGS, and their relative contributions to whole brain E4:E3 editing levels determined.

Correlation of Gene Editing with Protein Levels.

E4 and E3 protein will be measured in a sample of crude cell suspensions from each brain region by targeted MS [61, 62]; the stability of ApoE during this cell dissociation method will verified by spiking an E4 brain cell suspension with recombinant E3 protein (positive control). Protein samples will be reduced, alkylated, digested with trypsin and run through HPLC system to a triple quadrupole MS where multiple reaction monitoring acquisition retention times and fragment ion spectra for tryptic peptides specific for each ApoE isoform—LGADMEDVR (SEQ ID NO:19) for E4 and LGADMEDVCGR (SEQ ID NO:20) for E3 (aa #112 is bold). Peptides and pure E4 and E3 standards (BioVision Inc. recombinant E3 Cat #: 4696; PeproTech, Inc., recE4 Cat #350-05) with stable-isotope-labeled arginine will be used. Using this method, we will determine the vanishing E3 ratio in brain lysates spiked with recombinant E3 and E4 and determine the detection limit for E3.

### SE-CRISPR E4 to E3 Editing in Liver and Germ Line Tissues.

We will assess E4 to E3 editing in liver, testes, and ovaries of 7 day- and 4 month-treated mice. E3 and E4 in blood and liver will be correlated.

### Alternative Strategies:

To improve Tf surface presentation, Tf can be attached to the distal end of PEG-Pra, then linked to the lipid azide using Click. Tf effectiveness may be increased with the cell-penetrating ligand Penetratin [50]. PEG may interfere with SE release to the cytoplasm, which can be prevented by incorporation of endosomolytic moieties or using sheddable PEG coatings with cleavable linkages [52].

### Statistical Methods.

For all assays described herein, three or more groups will be compared by ANOVA with the Tukey or Kruskal-Wallis statistical analysis. As needed we will use the UCLA Biostatistical core group.

### Rigor, Reproducibility and Power Analysis.

The ApoE4 gene in the two biological models used herein, N2a-E4 cells and E4-TR mice, will be sequenced in the gRNA binding site, to verify they have the Reference Sequence, so the correct gRNA sequence is designed.

All in vitro analysis will be tested in triplicates per data point to get good CVs. Critical experiments will be repeated to ensure reproducibility. Because we will look at editing for the first time in E4-TR mice, power analysis at this time is not possible, however because we will look for an event that either does or does not occur—editing of E4 to E3—we believe an n=4 will be sufficient. Our studies will provide us with data on power analysis. Because the permeability of the BBB and other physiological parameters may be affected by gender, both male and female mice will be used.

### REFERENCES

[1] M. E. Belloy, V. Napolioni, M. D. Greicius, A Quarter Century of APOE and Alzheimer's Disease: Progress to Date and the Path Forward, Neuron, 101 (2019) 820-838. PMID: 30844401. PMC6407643.
[2] E. H. Corder, A. M. Saunders, W. J. Strittmatter, D. E. Schmechel, P. C. Gaskell, G. W. Small, A. D. Roses, J. L. Haines, M. A. Pericak-Vance, Gene dose of apolipoprotein E type 4 allele and the risk of Alzheimers disease in late onset families, Science (New York, N. Y.), 261 (1993) 921. PMID: 8346443.
[3] L. A. Farrer, L. A. Cupples, C. M. van Duijn, A. Kurz, R. Zimmer, U. Muller, R. C. Green, V. Clarke, J. Shoffner, D. C. Wallace, et al., Apolipoprotein E genotype in patients with Alzheimer's disease: implications for the risk of dementia among relatives, Annals of neurology, 38 (1995) 797-808. PMID: 7486872.
[4] C. Koriath, T. Lashley, W. Taylor, R. Druyeh, A. Dimitriadis, N. Denning, J. Williams, J. D. Warren, N. C. Fox, J. M. Schott, J. B. Rowe, J. Collinge, J. D. Rohrer, S. Mead, ApoE4 lowers age at onset in patients with frontotemporal dementia and tauopathy independent of amyloid-β copathology, Alzheimers Dement (Amst), 11 (2019) 277-280. PMID: 30949567. PMC6430720.
[5] Y. Shi, K. Yamada, S. A. Liddelow, S. T. Smith, L. Zhao, W. Luo, R. M. Tsai, S. Spina, L. T. Grinberg, J. C. Rojas, G. Gallardo, K. Wang, J. Roh, G. Robinson, M. B. Finn, H. Jiang, P. M. Sullivan, C. Baufeld, M. W. Wood, C. Sutphen, L. McCue, C. Xiong, J. L. Del-Aguila, J. C. Morris, C. Cruchaga, A. M. Fagan, B. L. Miller, A. L. Boxer, W. W. Seeley, O. Butovsky, B. A. Barres, S. M. Paul, D. M. Holtzman, ApoE4 markedly exacerbates tau-mediated neurodegeneration in a mouse model of tauopathy, Nature, 549 (2017) 523-527. PMID: 28959956. PMC5641217.
[6] J. Neitzel, N. Franzmeier, A. Rubinski, A. P. Binette, J. Poirier, S. Villeneuve, M. Ewers, P.-A. R. Group, I. the Alzheimer's Disease Neuroimaging, ApoE4 associated with higher tau accumulation independent of amyloid burden, Alzheimer's & Dementia, 16 (2020) e046206. PMID: no PMCID available.
[7] M. S. Baek, H. Cho, H. S. Lee, J. H. Lee, Y. H. Ryu, C. H. Lyoo, Effect of APOE &4 genotype on amyloid-β and tau accumulation in Alzheimer's disease, Alzheimers Res Ther, 12 (2020) 140. PMID: 33129364. PMC7603688.
[8] H. Braak, E. Braak, Neuropathological stageing of Alzheimer-related changes, Acta neuropathologica, 82 (1991) 239-259. PMID: 1759558.
[9] J. A. Hardy, G. A. Higgins, Alzheimer's disease: the amyloid cascade hypothesis, Science (New York, N. Y.), 256 (1992) 184-185. PMID: 1566067.
[10] C. Bancher, C. Brunner, H. Lassmann, H. Budka, K. Jellinger, G. Wiche, F. Seitelberger, I. Grundke-Iqbal, K. Iqbal, H. M. Wisniewski, Accumulation of abnormally phosphorylated t precedes the formation of neurofibrillary tangles in Alzheimer's disease, Brain research, 477 (1989) 90-99. PMID: 2495152.
[11] M. Goedert, Tau protein and the neurofibrillary pathology of Alzheimer's disease, Trends Neurosci, 16 (1993) 460-465. PMID: 7507619.
[12] J. K. Johnson, R. McCleary, M. H. Oshita, C. W. Cotman, Initiation and propagation stages of beta-amyloid are associated with distinctive apolipoprotein E, age, and gender profiles, Brain research, 798 (1998) 18-24. PMID: 9666062.
[13] R. W. Mahley, Y. Huang, Alzheimer disease: multiple causes, multiple effects of apolipoprotein E4, and multiple therapeutic approaches, Annals of neurology, 65 (2009) 623-625. PMID: 19557874.
[14] R. W. Mahley, Y. Huang, Small-molecule structure correctors target abnormal protein structure and function: structure corrector rescue of apolipoprotein E4-associated neuropathology, Journal of medicinal chemistry, 55 (2012) 8997-9008. PMID: 23013167. PMC4904786.
[15] A. S. Hanafy, S. Schoch, A. Lamprecht, CRISPR/Cas9 Delivery Potentials in Alzheimer's Disease Management: A Mini Review, Pharmaceutics, 12 (2020). PMID: 32854251. PMC7559557.
[16] S. He, The first human trial of CRISPR-based cell therapy clears safety concerns as new treatment for late-stage lung cancer, Signal Transduction and Targeted Therapy, 5 (2020) 168. PMID: 32843656 PMCID: PMC7447629.
[17] T. Hampton, With First CRISPR Trials, Gene Editing Moves Toward the Clinic, Jama, 323 (2020) 1537-1539. PMID: 32267478.
[18] N. Subbiah, J. Campagna, P. Spilman, M. P. Alam, S. Sharma, A. Hokugo, I. Nishimura, V. John, Deformable Nanovesicles Synthesized through an Adaptable Microfluidic Platform for Enhanced Localized Transdermal Drug Delivery, J Drug Deliv, 2017 (2017) 4759839. PMID: 28480080. PMC5396447.
[19] K. B. Johnsen, A. Burkhart, L. B. Thomsen, T. L. Andresen, T. Moos, Targeting the transferrin receptor for brain drug delivery, Prog Neurobiol, 181 (2019) 101665. PMID: 31376426.
[20] J. M. Rabanel, P. A. Piec, S. Landri, S. A. Patten, C. Ramassamy, Transport of PEGylated-PLA nanoparticles across a blood brain barrier model, entry into neuronal cells and in vivo brain bioavailability, J Control Release, 328 (2020) 679-695. PMID: 32979453.
[21] R. C. Klein, B. E. Mace, S. D. Moore, P. M. Sullivan, Progressive loss of synaptic integrity in human apolipoprotein E4 targeted replacement mice and attenuation by apolipoprotein E2, Neuroscience, 171 (2010) 1265-1272. PMID: 20951774.
[22] E. Genin, D. Hannequin, D. Wallon, K. Sleegers, M. Hiltunen, O. Combarros, M. J. Bullido, S. Engelborghs, P. De Deyn, C. Berr, F. Pasquier, B. Dubois, G. Tognoni, N. Fievet, N. Brouwers, K. Bettens, B. Arosio, E. Coto, M. Del Zompo, I. Mateo, J. Epelbaum, A. Frank-Garcia, S. Helisalmi, E. Porcellini, A. Pilotto, P. Forti, R. Ferri, E. Scarpini, G. Siciliano, V. Solfrizzi, S. Sorbi, G. Spalletta, F. Valdivieso, S. Vepsäläinen, V. Alvarez, P. Bosco, M. Mancuso, F. Panza, B. Nacmias, P. Bossù, O. Hanon, P. Piccardi, G. Annoni, D. Seripa, D. Galimberti, F. Licastro, H. Soininen, J. F. Dartigues, M. I. Kamboh, C. Van Broeckhoven, J. C. Lambert, P. Amouyel, D. Campion, APOE and Alzheimer disease: a major gene with semi-dominant inheritance, Mol Psychiatry, 16 (2011) 903-907. PMID: 21556001. PMC3162068.
[23] J. Cao, F. E. Gaamouch, J. S. Meabon, K. D. Meeker, L. Zhu, M. B. Zhong, J. Bendik, G. Elder, P. Jing, J. Xia, W. Luo, D. G. Cook, D. Cai, ApoE4-associated phospholipid dysregulation contributes to development of Tau hyper-phosphorylation after traumatic brain injury, Scientific reports, 7 (2017) 11372. PMID: 28900205. PMC5595858.
[24] C. C. Liu, N. Zhao, Y. Fu, N. Wang, C. Linares, C. W. Tsai, G. Bu, ApoE4 Accelerates Early Seeding of Amyloid Pathology, Neuron, 96 (2017) 1024-1032.e1023. PMID: 29216449.
[25] R. W. Mahley, Y. Huang, K. H. Weisgraber, Detrimental effects of apolipoprotein E4: potential therapeutic targets in Alzheimer's disease, Current Alzheimer research, 4 (2007) 537-540. PMID: 18220516.
[26] R. Vassar, Seeds of Destruction: New Mechanistic Insights into the Role of Apolipoprotein E4 in Alzheimer's Disease, Neuron, 96 (2017) 953-955. PMID: 29216455.
[27] A. Serrano-Pozo, S. Das, B. T. Hyman, APOE and Alzheimer's disease: advances in genetics, pathophysiology, and therapeutic approaches, The Lancet. Neurology, 20 (2021) 68-80. PMID: 33340485. PMC8096522.
[28] M. Xiong, H. Jiang, J. R. Serrano, E. R. Gonzales, C. Wang, M. Gratuze, R. Hoyle, N. Bien-Ly, A. P. Silverman, P. M. Sullivan, R. J. Watts, J. D. Ulrich, G. J. Zipfel, D. M. Holtzman, APOE immunotherapy reduces cerebral amyloid angiopathy and amyloid plaques while improving cerebrovascular function, Science translational medicine, 13 (2021). PMID: 33597265.
[29] C. Wang, R. Najm, Q. Xu, D.-e. Jeong, D. Walker, M. E. Balestra, S. Y. Yoon, H. Yuan, G. Li, Z. A. Miller, B. L. Miller, M. J. Malloy, Y. Huang, Gain of toxic apolipoprotein E4 effects in human iPSC-derived neurons is ameliorated by a small-molecule structure corrector, Nature medicine, 24 (2018) 647-657. PMID: 29632371 PMCID: PMC5948154.
[30] A. C. Komor, Y. B. Kim, M. S. Packer, J. A. Zuris, D. R. Liu, Programmable editing of a target base in genomic DNA without double-stranded DNA cleavage, Nature, 533 (2016) 420-424. PMID: 27096365. PMC4873371.
[31] T. T. Rohn, N. Kim, N. F. Isho, J. M. Mack, The Potential of CRISPR/Cas9 Gene Editing as a Treatment Strategy for Alzheimer's Disease, J Alzheimers Dis Parkinsonism, 8 (2018). PMID: 30090689. PMC6078432.
[32] M. Safieh, A. D. Korczyn, D. M. Michaelson, ApoE4: an emerging therapeutic target for Alzheimer's disease, BMC Medicine, 17 (2019) 64. PMID: 30890171 PMCID: PMC6425600.
[33] K. H. Weisgraber, Apolipoprotein E distribution among human plasma lipoproteins: role of the cysteine-arginine interchange at residue 112, J Lipid Res, 31 (1990) 1503-1511. PMID: 2280190.
[34] A. R. Wadhwani, A. Affaneh, S. Van Gulden, J. A. Kessler, Neuronal apolipoprotein E4 increases cell death and phosphorylated tau release in alzheimer disease, Annals of neurology, 85 (2019) 726-739. PMID: 30840313. PMC8123085.
[35] Y. T. Lin, J. Seo, F. Gao, H. M. Feldman, H. L. Wen, J. Penney, H. P. Cam, E. Gjoneska, W. K. Raja, J. Cheng, R. Rueda, O. Kritskiy, F. Abdurrob, Z. Peng, B. Milo, C. J. Yu, S. Elmsaouri, D. Dey, T. Ko, B. A. Yankner, L. H. Tsai, APOE4 Causes Widespread Molecular and Cellular Alterations Associated with Alzheimer's Disease Phenotypes in Human iPSC-Derived Brain Cell Types, Neuron, 98 (2018) 1294. PMID: 29953873. PMC6048952.
[36] F. Caso, B. Davies, Base editing and prime editing in laboratory animals, Laboratory animals, (2021) 23677221993895. PMID: 33596731. PMID: 33596731.
[37] C. A. Vasquez, Q. T. Cowan, A. C. Komor, Base Editing in Human Cells to Produce Single-Nucleotide-Variant Clonal Cell Lines, Current protocols in molecular biology, 133 (2020) e129. PMID: 33151638. PMC7654285.
[38] W. H. Yeh, H. Chiang, H. A. Rees, A. S. B. Edge, D. R. Liu, In vivo base editing of post-mitotic sensory cells, Nat Commun, 9 (2018) 2184. PMID: 29872041. PMC5988727 Pairwise Plants, companies that are using genome editing. A. S. B. E. is a consultant and co-founder of Decibel Therapeutics. The remaining authors declare no competing interests.
[39] P. Antoniou, A. Miccio, M. Brusson, Base and Prime Editing Technologies for Blood Disorders, Frontiers in Genome Editing, 3 (2021). PMID: no PMCID available.
[40] K. Saha, E. J. Sontheimer, P. J. Brooks, M. R. Dwinell, C. A. Gersbach, D. R. Liu, S. A. Murray, S. Q. Tsai, R. C. Wilson, D. G. Anderson, A. Asokan, J. F. Banfield, K. S. Bankiewicz, G. Bao, J. W. M. Bulte, N. Bursac, J. M. Campbell, D. F. Carlson, E. L. Chaikof, Z. Y. Chen, R. H. Cheng, K. J. Clark, D. T. Curiel, J. E. Dahlman, B. E. Deverman, M. E. Dickinson, J. A. Doudna, S. C. Ekker, M. E. Emborg, G. Feng, B. S. Freedman, D. M. Gamm, G. Gao, I. C. Ghiran, P. M. Glazer, S. Gong, J. D. Heaney, J. D. Hennebold, J. T. Hinson, A. Khvorova, S. Kiani, W. R. Lagor, K. S. Lam, K. W. Leong, J. E. Levine, J. A. Lewis, C. M. Lutz, D. H. Ly, S. Maragh, P. B. McCray, Jr., T. C. McDevitt, O. Mirochnitchenko, R. Morizane, N. Murthy, R. S. Prather, J. A. Ronald, S. Roy, S. Roy, V. Sabbisetti, W. M. Saltzman, P. J. Santangelo, D. J. Segal, M. Shimoyama, M. C. Skala, A. F. Tarantal, J. C. Tilton, G. A. Truskey, M. Vandsburger, J. K. Watts, K. D. Wells, S. A. Wolfe, Q. Xu, W. Xue, G. Yi, J. Zhou, The NIH Somatic Cell Genome Editing program, Nature, 592 (2021) 195-204. PMID: 33828315. PMC8026397.
[41] C. Peters-Libeu, J. Campagna, M. Mitsumori, K. S. Poksay, P. Spilman, A. Sabogal, D. E. Bredesen, V. John, sAbetaPPalpha is a Potent Endogenous Inhibitor of BACE1, Journal of Alzheimer's disease: JAD, 47 (2015) 545-555. PMID: 26401691.
[42] K. Labun, T. G. Montague, M. Krause, Y. N. Torres Cleuren, H. Tjeldnes, E. Valen, CHOPCHOP v3: expanding the CRISPR web toolbox beyond genome editing, Nucleic Acids Research, 47 (2019) W171-W174. PMID: 31106371 PMCID: PMC6602426.
[43] Synthego, ICE v2 CRISPR Tool, Synthego, (2021). PMID: www.synthego.com/products/bioinformatics/crispr-analysis.
[44] H. Nishimasu, X. Shi, S. Ishiguro, L. Gao, S. Hirano, S. Okazaki, T. Noda, O. O. Abudayyeh, J. S. Gootenberg, H. Mori, S. Oura, B. Holmes, M. Tanaka, M. Seki, H. Hirano, H. Aburatani, R. Ishitani, M. Ikawa, N. Yachie, F. Zhang, O. Nureki, Engineered CRISPR-Cas9 nuclease with expanded targeting space, Science (New York, N. Y.), 361 (2018) 1259-1262. PMID: 30166441. PMC6368452.
[45] crisprbehive, design, BE HIve, www.crisprbehive.design, (2021). PMID: no PMID.
[46] M. Arbab, M. W. Shen, B. Mok, C. Wilson, Ż. Matuszek, C. A. Cassa, D. R. Liu, Determinants of Base Editing Outcomes from Target Library Analysis and Machine Learning, Cell, 182 (2020) 463-480.e430. PMID: 32533916. PMC7384975.
[47] Moriarty, EditR Moriarty Lab University of Minnesota, https://moriaritylab.shinyapps.io/editr_v10/(2021). PMID: no PMID.
[48] Pinello, Pinello Lab CRISRPESSO, www.crispresso.pinellolab.partners.org, (2021). PMID: no PMID.
[49] P. D. Hsu, D. A. Scott, J. A. Weinstein, F. A. Ran, S. Konermann, V. Agarwala, Y. Li, E. J. Fine, X. Wu, O. Shalem, T. J. Cradick, L. A. Marraffini, G. Bao, F. Zhang, DNA targeting specificity of RNA-guided Cas9 nucleases, Nature biotechnology, 31 (2013) 827-832. PMID: 23873081. PMC3969858.
[50] B. Dos Santos Rodrigues, T. Kanekiyo, J. Singh, ApoE-2 Brain-Targeted Gene Therapy Through Transferrin and Penetratin Tagged Liposomal Nanoparticles, Pharm Res, 36 (2019) 161. PMID: 31529284.
[51] S. A. A. Kooijmans, L. A. L. Fliervoet, R. van der Meel, M. Fens, H. F. G. Heijnen, P. M. P. van Bergen En Henegouwen, P. Vader, R. M. Schiffelers, PEGylated and targeted extracellular vesicles display enhanced cell specificity and circulation time, J Control Release, 224 (2016) 77-85. PMID: 26773767.
[52] J. S. Suk, Q. Xu, N. Kim, J. Hanes, L. M. Ensign, PEGylation as a strategy for improving nanoparticle-based drug and gene delivery, Advanced drug delivery reviews, 99 (2016) 28-51. PMID: 26456916. PMC4798869.
[53] E. Nance, K. F. Timbie, G. Miller, J. Song, C. Louttit, A. Klibanov, T.-Y. Shih, G. Swaminathan, R. Tamargo, G. Woodworth, J. Hanes, R. Price, Non-invasive delivery of stealth, brain-penetrating nanoparticles across the blood-brain barrier using MRI-guided focused ultrasound, J Control Release, 189 (2014) 123-132. PMID: 24979210 PMCID: PMC4125545.
[54] F. Ordikhani, N. Zandi, M. Mazaheri, G. A. Luther, M. Ghovvati, A. Akbarzadeh, N. Annabi, Targeted nanomedicines for the treatment of bone disease and regeneration, Medicinal research reviews, 41 (2021) 1221-1254. PMID: 33347711.
[55] E. A. Nance, G. F. Woodworth, K. A. Sailor, T. Y. Shih, Q. Xu, G. Swaminathan, D. Xiang, C. Eberhart, J. Hanes, A dense poly(ethylene glycol) coating improves penetration of large polymeric nanoparticles within brain tissue, Science translational medicine, 4 (2012) 149ra119. PMID: 22932224. PMC3718558.
[56] H. C. Kolb, M. G. Finn, K. B. Sharpless, Click Chemistry: Diverse Chemical Function from a Few Good Reactions, Angewandte Chemie (International ed. in English), 40 (2001) 2004-2021. PMID: 11433435.
[57] Q. Wang, T. R. Chan, R. Hilgraf, V. V. Fokin, K. B. Sharpless, M. G. Finn, Bioconjugation by copper(I)-catalyzed azide-alkyne [3+2] cycloaddition, Journal of the American Chemical Society, 125 (2003) 3192-3193. PMID: 12630856.
[58] A. Montagne, D. A. Nation, A. P. Sagare, G. Barisano, M. D. Sweeney, A. Chakhoyan, M. Pachicano, E. Joe, A. R. Nelson, L. M. D'Orazio, D. P. Buennagel, M. G. Harrington, T. L. S. Benzinger, A. M. Fagan, J. M. Ringman, L. S. Schneider, J. C. Morris, E. M. Reiman, R. J. Caselli, H. C. Chui, J. Tcw, Y. Chen, J. Pa, P. S. Conti, M. Law, A. W. Toga, B. V. Zlokovic, APOE4 leads to blood-brain barrier dysfunction predicting cognitive decline, Nature, 581 (2020) 71-76. PMID: 32376954. PMC7250000.
[59] J. Satoh, Y. Kino, N. Asahina, M. Takitani, J. Miyoshi, T. Ishida, Y. Saito, TMEM119 marks a subset of microglia in the human brain, Neuropathology, 36 (2016) 39-49. PMID: 26250788.
[60] M. L. Bennett, F. C. Bennett, S. A. Liddelow, B. Ajami, J. L. Zamanian, N. B. Fernhoff, S. B. Mulinyawe, C. J. Bohlen, A. Adil, A. Tucker, I. L. Weissman, E. F. Chang, G. Li, G. A. Grant, M. G. Hayden Gephart, B. A. Barres, New tools for studying microglia in the mouse and human CNS, Proceedings of the National Academy of Sciences of the United States of America, 113 (2016) E1738-1746. PMID: 26884166. PMC4812770.
[61] C. Hirtz, J. Vialaret, G. Nouadje, S. Schraen, P. Benlian, S. Mary, P. Philibert, L. Tiers, P. Bros, C. Delaby, A. Gabelle, S. Lehmann, Development of new quantitative mass spectrometry and semi-automatic isofocusing methods for the determination of Apolipoprotein E typing, Clinica chimica acta; international journal of clinical chemistry, 454 (2016) 33-38. PMID: 26707914.
[62] T. West, K. M. Kirmess, M. R. Meyer, M. S. Holubasch, S. S. Knapik, Y. Hu, J. H. Contois, E. N. Jackson, S. E. Harpstrite, R. J. Bateman, D. M. Holtzman, P. B. Verghese, I. Fogelman, J. B. Braunstein, K. E. Yarasheski, A blood-based diagnostic test incorporating plasma Aβ42/40 ratio, ApoE proteotype, and age accurately identifies brain amyloid status: findings from a multi cohort validity analysis, Mol Neurodegener, 16 (2021) 30. PMID: 33933117. PMC8088704.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A synthetic exosome capable of delivering a therapeutic moiety across the blood brain barrier into the central nervous system (CNS), said synthetic exosome comprising:
a liposome formed from a lipid bilayer, where said lipid bilayer comprises:
one or more phospholipids selected from the group consisting of phosphate lipids, phosphoglycerol lipids, phosphocholine lipids, and phosphoethanolamine lipids where the lipid carbon chain ranges from 3 to 24 carbon atoms;
cholesterol, a cholesterol derivative, or a phytosterol; and
a non-ionic surfactant;
wherein said lipid bilayer does not contain an alcohol;
said exosome is less than about 200 nm in diameter; and
wherein said synthetic exosome contains a guide RNA (gRNA) and a cytidine base editor, where said gRNA comprise a sequence that directs said n-Cas9-cytidine deaminase to edit the codon for amino acid 112 of the gene encoding ApoE4, and/or said synthetic exosome contains one or more proteins selected from the group consisting of SE-sAPPα, iduronidase (IDUA), SE(ζ−)-IDUA, SE(ζ+)-IDUA, acid sphingomylienase (ASM), and SE-ASM.

2. The synthetic exosome of claim 1, wherein said synthetic exosome contains a guide RNA (gRNA) and a cytidine base editor, where said gRNA comprise a sequence that directs said n-Cas9-cytidine deaminase to edit the codon for amino acid 112 of the gene encoding ApoE4.

3. The synthetic exosome of claim 2, wherein said cytidine base editor comprises comprising an nCas9-cytidine deaminase.

4. The synthetic exosome of claim 3, wherein said cytidine base editor selected from the group consisting of BE3, BE4, BE4max, and AncBE4max.

5. The synthetic exosome of claim 4, wherein said cytidine base editor comprises BE3.

6. The synthetic exosome of claim 4, wherein said cytidine base editor comprises BE4.

7. The synthetic exosome of claim 4, wherein said cytidine base editor comprises BE4max.

8. The synthetic exosome of claim 4, wherein said cytidine base editor comprises AncBE4max.

9. The synthetic exosome according to any one of claims 3-8, wherein said guide RNA is complexed to said nCas9-cytidine deaminase.

10. The synthetic exosome according to any one of claims 1-9, wherein the gRNA sequence comprises the same 20 nt sequence as the protospacer sequence abutting the PAM as shown in Table 1.

11. The synthetic exosome according to any one of claims 1-9, wherein the gRNA sequence comprises the same sequence as the protospacer sequence abutting the PAM as shown in Table 2.

12. The synthetic exosome according to any one of claims 1-9, wherein the gRNA sequence comprises or consists of the crRNA sequence GGACGUGCGCGGCCGCCUGG (SEQ ID NO:17).

13. The synthetic exosome according to any one of claims 10-12, wherein said gRNA further comprises a tracrRNA that specifically binds Cas9 protein.

14. The synthetic exosome of claim 13, wherein said tracrRNA comprises an 80 nt sequence.

15. The synthetic exosome of claim 13, wherein said tracrRNA sequence comprises or consists of the sequence AGCAUAGCAAGUUUAAAUAAGGCUAGUC CGUUAUCAACUUGAAAAAGUGGCACCGAGUCGGUGCUUU (SEQ ID NO:18).

16. The synthetic exosome according to any one of claims 12-15, wherein said gRNA sequence comprises a linker RNA joining said tracrRNA sequence to said crRNA sequence.

17. The synthetic exosome of claim 16, wherein said linker RNA ranges in length from about 6 nt or from about 8 nt up to about 20 nt, or up to about 16 nt, or up to about 12 nt, or up to about 10 nt.

18. The synthetic exosome of claim 17, wherein said linker comprises a sequence complementary to a region of said tracrRNA.

19. The synthetic exosome according to any one of claims 1-16, wherein the ratio of Cas9 to gRNA in said exosome ranges from about 0.5 to 2 to about 2 to 0.5.

20. The synthetic exosome of claim 19, wherein the ratio of Cas9 to gRNA in said exosome ranges from about 1:1 to about 2:1.

21. The synthetic exosome of claim 20, wherein the ratio of Cas9 to gRNA in said exosome is about 1.5:1.

22. The synthetic exosome of claim 1, wherein said exosome is about 50 nm up to about 200 nm in diameter, or about 50 nm up to about 150 nm in diameter, or about 50 nm up to about 110 nm.

23. The synthetic exosome according to any one of claims 1-21, wherein said exosome is less than about 150 nm in diameter.

24. The synthetic exosome according to any one of claim 1-23, wherein said synthetic exosome is capable of crossing the blood brain barrier without substantially leaking said nCas9-cytidine deaminase and/or said gRNA.

25. The synthetic exosome according to any one of claims 1-24, wherein said lipid bilayer consists of said one or more phospholipids, said cholesterol or cholesterol derivative or a phytosterol; and said non-ionic surfactant.

26. The synthetic exosome according to any one of claims 1-25, wherein said exosome is capable of crossing the blood/brain barrier (BBB) and delivering the nCas9-cytidine deaminase and gRNA into a brain cell in sufficient amount to edit the genome of said brain cell.

27. The synthetic exosome of claim 26, wherein said exosome is capable of crossing the blood/brain barrier (BBB) and delivering the nCas9-cytidine deaminase and gRNA contained therein to the central nervous system without losing more than about 40%, or without losing more than 30%, or without losing more than 20%, or without losing more than 10%, or without losing more than 5%, or without losing more than 3%, or without losing more than 1% of the nCas9-cytidine deaminase and gRNA.

28. The synthetic exosome of claim 1, wherein said synthetic exosome contains one or more proteins selected from the group consisting of SE-sAPPα, iduronidase (IDUA), SE(ζ−)-IDUA, SE(ζ+)-IDUA, acid sphingomylienase (ASM), and SE-ASM.

29. The synthetic exosome of claim 28, wherein said synthetic exosome contains one or more proteins selected from the group consisting of SE-sAPPα.

30. The synthetic exosome according to any one of claims of claim 28-29, wherein said synthetic exosome contains iduronidase (IDUA).

31. The synthetic exosome according to any one of claims of claim 28-30, wherein said synthetic exosome contains SE(ζ−)-IDUA.

32. The synthetic exosome according to any one of claims of claim 28-31, wherein said synthetic exosome contains SE(ζ+)-IDUA.

33. The synthetic exosome according to any one of claims of claim 28-32, wherein said synthetic exosome contains acid sphingomylienase (ASM).

34. The synthetic exosome according to any one of claims of claim 28-33, wherein said synthetic exosome contains SE-ASM.

35. The synthetic exosome according to any one of claims 1-41, wherein said lipid bilayer does not contain an alcohol.

36. The synthetic exosome of claim 35, wherein said lipid bilayer does not contain ethanol.

37. The synthetic exosome according to any one of claims 1-36, wherein said bilayer does not contain glutathione-maleimide-PEG2000-distearoyl phosphatidyl ethanolamine.

38. The synthetic exosome according to any one of claims 1-37, wherein said exosome is not a transferosome.

39. The synthetic exosome according to any one of claims 1-38, wherein said exosome is not an ethosome.

40. The synthetic exosome according to any one of claims 1-39, wherein the molar ratio of total phospholipid to cholesterol, cholesterol, or phytosterol ranges from about 6-10 moles of total phospholipid to about 1-3 moles of cholesterol.

41. The synthetic exosome according to any one of claims 1-40, wherein the amount of surfactant ranges from about 1%, or from about 3%, or from about 5%, or from about 8% up to about 18%, or up to about 15%, or up to about 13%, or up to about 10% (wt/wt).

42. The synthetic exosome according to any one of claims 1-41, wherein said surfactant comprise one or more surfactants selected from the group consisting of Span 80, Tween 20, BRIJ® 76 (stearyl poly(10)oxy ethylene ether), BRIJ® 78 (stearyl poly(20)oxyethylene ether), BRIJ® 96 (oleyl poly(10)oxy ethylene ether), and BRIJ® 721 (stearyl poly (21) oxyethylene ether).

43. The synthetic exosome of claim 42, wherein said surfactant comprises or consists of Span 80.

44. The synthetic exosome of claim 43, wherein the lipid bilayer comprises about 10% to about 20%, or about 15% Span 80 by weight.

45. The synthetic exosome according to any one of claims 1-44, wherein said cholesterol, cholesterol derivative, or phytosterol comprises or consists of cholesterol.

46. The synthetic exosome according to any one of claims 1-44, wherein said cholesterol, cholesterol derivative, or phytosterol comprises or consists of a cholesterol derivative selected from the group consisting of cholesterol hemisuccinate, lysine-based cholesterol (CHLYS), 20-hydroxychloesterol, 22-hydroxycholesterol, 24-hydroxycholesterol, 25-hydroxy cholesterol, 27-hydroxycholesterol, cholesteryl succinate, cholic succinate, cholic tri-succinate, lithocholic succinate, chenodesoxycholic bis-scuccinate, and Hederoside.

47. The synthetic exosome according to any one of claims 1-44, wherein said cholesterol, cholesterol derivative comprises or consists cholesterol hemisuccinate.

48. The synthetic exosome according to any one of claims 1-44, wherein said cholesterol, cholesterol derivative, or phytosterol comprises or consists of a phytosterol.

49. The synthetic exosome of claim 48, wherein said phytosterol comprises a 9,10-secosteroid.

50. The synthetic exosome of claim 49, wherein said 9,10 secosteroid comprises a compound selected from the group consisting of vitamin D3, vitamin D2, calcipotriol.

51. The synthetic exosome of claim 48, wherein said phytosterol comprises a C-24 alkyl steroid.

52. The synthetic exosome of claim 51, wherein said C-24 alkyl steroid comprises a compound selected from the group consisting of stigmasterol, and β-sitosterol.

53. The synthetic exosome of claim 48, wherein said phytosterol comprises a pentacyclic steroid.

54. The synthetic exosome of claim 53, wherein said pentacyclic steroid comprises a compound selected from the group consisting of betulin, lupeol, ursolic acid, and oleanolic acid.

55. The synthetic exosome according to any one of claims 1-54, wherein said cholesterol, cholesterol derivative, or phytosterol is pegylated.

56. The synthetic exosome according to any one of claims 1-55, wherein said one or more phospholipids comprises one or more phospholipids selected from the group consisting of dihexanoyl-sn-glycero-3-phosphate (DHPA), didecanoyl-sn-glycero-3-phosphate (DDPA), distearoyl-sn-glycero-3-phosphate (DTPA), and dihexadecyl phosphate (DHP).

57. The synthetic exosome according to any one of claims 1-56, wherein said one or more phospholipids comprises one or more phosphoglycerol lipids selected from the group consisting of dihexanoyl-sn-glycero-3-phospho-(1′-rac-glycerol) (DHPG), dilauroyl-sn-glycero-3-phospho-(1′-rac-glycerol) (DLPG), and distearoyl-sn-glycero-3-phospho-(1′-rac-glycerol) (DTPG).

58. The synthetic exosome according to any one of claims 1-57, wherein said one or more phospholipids comprises one or more phosphocholine lipids selected from the group consisting of dipropionyl-sn-glycero-3-phosphocholine (PC), diheptanoyl-sn-glycero-3-phosphocholine (DHPC), dimyristoyl-sn-glycero-3-phosphocholine (DMPC), and dilignoceroyl-sn-glycero-3-phosphocholine (DGPC).

59. The synthetic exosome according to any one of claims 1-57, wherein said one or more phospholipids comprises one or more phosphoethanolamine lipids selected from the group consisting of sihexanoyl-sn-glycero-3-phosphoethanolamine (DHPE), and distearoyl-sn-glycero-3-phosphoethanolamine (DTPE).

60. The synthetic exosome according to any one of claims 1-57, wherein said one or more phospholipids comprises one or more phosphoethanolamine-PEG lipids selected from the group consisting of dipalmitoyl-sn-glycero-3-phospho(ethylene glycol) (DPPEG1), dimyristoyl-sn-glycero-3-phosphoethanolamine-N-[methoxy(polyethylene glycol)-350 (DMPEG350), distearoyl-sn-glycero-3-phosphoethanolamine-N-[methoxy(polyethylene glycol)-350] (DTPEG350), dimyristoyl-sn-glycero-3-phosphoethanolamine-N-[methoxy(polyethylene glycol)-550] (DMPEG550), and dimyristoyl-sn-glycero-3-phosphoethanolamine-N-[methoxy(polyethylene glycol)-1000] (DMPEG1000).

61. The synthetic exosome according to any one of claims 1-57, wherein said one or more phospholipids comprises one or more phospholipids selected from the group consisting of dioleoyl-sn-glycero-3-phosphocholine (N-aminoethyl) (PC-NH₂), diphytanoyl-sn-glycero-3-phosphoethanolamine, dioleoyl-3-trimethylammonium-propane (DOTAP), distearoyl-3-trimethylammonium-propane (DSTAP), dimyristoyl-3-trimethylammonium-propane (DMTAP), and di-O-octadecyl-sn-glycero-3-phosphocholin (DOPC).

62. The synthetic exosome according to any one of claims 1-61, wherein said one or more phospholipids is functionalized with a targeting moiety selected from the group consisting of transferrin, an amino acid, a blood brain barrier targeting antibody, insulin, folic acid, and low density lipoprotein receptor related protein 1.

63. The synthetic exosome according to any one of claims 1-62, wherein said lipid bilayer comprises or consists of:
said surfactant; and
3:2:1 molar ratio (DHPA:DHP:CH), 1:5:1 molar ratio (DHPG:DHPA:CH), 2:5:1:2 molar ratio (DHPG:DHPA:PC-NH2:CH), or 2:4:1:1:2 molar ratio (DHPG:DHPA:PC-NH2:DMPEG350:CH) to provide synthetic exosomes having a zeta potential of about −20 mV or lower.

64. The synthetic exosome according to any one of claims 1-62, wherein said lipid bilayer comprises or consists of:
said surfactant; and
2:2:1 molar ratio (DHPG:DHPC:CH), 4:4:1:2 molar ratio (DHPG:DHPA:PC-NH2:CH), 2:2:1 molar ratio (DHPG:DHPA:CH), 4:4:1:1:2 molar ratio (DHPG:DHPA:PC-NH2:DMPEG550:CH), 2:2:1 molar ratio (DHPG:DTPE:CH), 2:2:1 molar ratio (DHPG:DMTAP:CH), 4:4:1:2 molar ratio (DHPG:DMTAP:PC-NH2:CH), or 4:4:1:1:2 molar ratio (DHPG:DMTAP:PC-NH2:DMPEG550:CH) to provide synthetic exosomes having a zeta potential ranging from about −20 mV to about 20 mV.

65. The synthetic exosome according to any one of claims 1-62, wherein said lipid bilayer comprises or consists of:
said surfactant; and 2:4:1 molar ratio (DHPC:DTPE:CH), 2:4:1 molar ratio (DHPC:DOTAP:CH), 2:4:1:2 molar ratio (DHPC:DMTAP:PC-NH2:CH), or 2:4:1:1:2 molar ratio (DHPC:DMTAP:PC-NH2:DMPEG350:CH) to provide synthetic exosomes having a zeta potential of about 20 mV or greater.

66. The synthetic exosome according to any one of claims 1-62, wherein said lipid bilayer comprises or consists of:
said surfactant; and 4:2:1 molar ratio (DTPA:DHP:CH), 1:5:1 molar ratio (DTPG:DTPA:CH), 1:5:1:2 molar ratio (DTPG:DTPA:PC-NH2:CH), or 1:4:1:1:2 molar ratio (DTPG:DTPA:PC-NH2:DMPEG350:CH) to provide synthetic exosomes having a zeta potential of about −20 mV or lower.

67. The synthetic exosome according to any one of claims 1-62, wherein said lipid bilayer comprises or consists of:
said surfactant; and 2:2:1 molar ratio (DTPG:DGPC:CH), 4:4:1:2 molar ratio (DTPG:DDPA:PC-NH2:CH), 2:2:1 molar ratio (DTPG:DDPA:CH), 4:4:1:1:2 molar ratio (DTPG:DDPA:PC-NH2:DMPEG550:CH), 2:2:1 molar ratio (DTPG:DTPE:CH), 2:2:1 molar ratio (DTPG:DMTAP:CH), 4:4:1:2 molar ratio (DTPG:DMTAP:PC-NH2:CH), or 4:4:1:1:2 molar ratio (DTPG:DMTAP:PC-NH2:DMPEG550:CH) to provide synthetic exosomes having a zeta potential ranging from about −20 mV to about 20 mV.

68. The synthetic exosome according to any one of claims 1-62, wherein said lipid bilayer comprises or consists of:
said surfactant; and 2:4:1 molar ratio (DMPC:DTPE:CH), 2:4:1 molar ratio (DMPC:DOTAP:CH), 2:4:1:2 molar ratio (DMPC:DMTAP:PC-NH2:CH), or 2:4:1:1:2 molar ratio (DMPC:DMTAP:PC-NH2:DMPEG350:CH) to provide synthetic exosomes having a zeta potential of about 20 mV or greater.

69. The synthetic exosome according to any one of claims 63-68, wherein CH is cholesterol.

70. The synthetic exosome according to any one of claims 63-68, wherein CH is a cholesterol derivative.

71. The synthetic exosome of claim 70, wherein said cholesterol derivative is selected from the group consisting of cholesterol hemisuccinate, lysine-based cholesterol (CHLYS), 20-hydroxychloesterol, 22-hydroxycholesterol, 24-hydroxycholesterol, 25-hydroxy cholesterol, 27-hydroxycholesterol, cholesteryl succinate, cholic succinate, cholic tri-succinate, lithocholic succinate, chenodesoxycholic bis-scuccinate, and Hederoside.

72. The synthetic exosome of claim 71, wherein CH is cholesterol hemisuccinate.

73. The synthetic exosome according to any one of claims 63-68, wherein CH is a phytosterol.

74. The synthetic exosome of claim 73, wherein CH is a C-24 alkyl steroid.

75. The synthetic exosome of claim 73, wherein CH is a C-24 alkyl steroid.

76. The synthetic exosome according to any one of claims 1-75, wherein a targeting moiety comprising or consisting of transferrin or transferrin receptor binding peptides, or folic acid, or an amino acid, or insulin, or a low density lipoprotein receptor related protein 1 is attached to said exosome.

77. The synthetic exosome according to any one of claims 1-75, wherein a targeting moiety comprising or consisting of a blood brain barrier targeting antibody is attached to said exosome.

78. The synthetic exosome according to any one of claims 1-75, wherein said exosome is attached to an antibody or a ligand that binds to a moiety selected from the group consisting of a transferrin receptor, an insulin receptor, an insulin growth factor receptor (IGFIR), a low-density lipoprotein (LDL) receptor, basigin, Glut1, CD98hc, and TMEM30A(cdc50A).

79. The synthetic exosome according to any one of claims 1-75, wherein said exosome is attached to an antibody or a ligand that binds to a cell surface marker.

80. The synthetic exosome of claim 79, wherein said cell surface marker is a marker of neural or glial cells.

81. The synthetic exosome of claim 79, wherein said cell surface marker is selected from the group consisting of CD63, CD81, CD9, and CD171, and is incorporated in the lipid bilayer of said exosome.

82. The synthetic exosome according to any one of claims 1-81, wherein a said synthetic exosome is pegylated.

83. A pharmaceutical formulation comprising:
a synthetic exosome according to any one of claims 1-82; and
a pharmaceutically acceptable carrier.

84. The formulation of claim 83, wherein said formulation is compounded for delivery by route selected from the group consisting of oral delivery, isophoretic delivery, subdermal delivery, transdermal delivery, parenteral delivery, aerosol administration, administration via inhalation, intravenous administration, and rectal administration.

85. The formulation of claim 84, wherein said formulation is compounded for oral administration.

86. The formulation of claim 84, wherein said formulation is compounded for transdermal administration.

87. The formulation of claim 86, wherein said formulation is provided as a transdermal patch.

88. The formulation of claim 84, wherein said formulation is compounded for systemic administration.

89. The formulation according to any one of claims 83-88, wherein said formulation is a unit dosage formulation.

90. A kit comprising a container containing a synthetic exosome according to any one of claims 1-82, and/or a pharmaceutical formulation according to any one of claims 83-89.

91. The kit of claim 90, wherein said kit comprises instructional materials teaching the use of said synthetic exosome to mitigate one or more symptoms associated with a disease characterized by the presence of and/or elevated presence of Apoe4, and/or the use of said composition in delaying or preventing the onset of one or more of said symptoms of said disease.

92. A method of reducing the risk, lessening the severity, or delaying the progression or onset of a pathology characterized by the presence of and/or elevated presence of Apoe4 in the brain of a mammal, said method comprising:
administering or causing to be administered, to said mammal synthetic exosome according to any one of claims 1-82, and/or a pharmaceutical formulation according to any one of claims 83-89 in an amount sufficient to reduce the risk, lessen the severity, or delay the progression or onset of said disease.

93. The method of claim 92, wherein said pathology comprises a pathology selected from the group consisting of Alzheimer's disease (AD), frontotemporal dementia, cerebral amyloid angiopathy (CAA), dementia with Lewy bodies (DLB), tauopathy, cerebrovascular disease, multiple sclerosis, vascular dementia, and traumatic brain injury hemorrhage, Parkinson's disease, Age related macular degeneration (AMD), dementia after stroke, cardiovascular disease, and/or LDL levels.

94. A method of preventing or delaying the onset of a pre-Alzheimer's condition and/or cognitive dysfunction, and/or ameliorating one or more symptoms of a pre-Alzheimer's condition and/or cognitive dysfunction, or preventing or delaying the progression of a pre-Alzheimer's condition or cognitive dysfunction to Alzheimer's disease in a mammal, said method comprising:
administering, or causing to be administered, to said mammal an effective amount of a synthetic exosome according to any one of claims 1-82, and/or a pharmaceutical formulation according to any one of claims 83-89.

95. A method of treating a pathology in a mammal selected from the group consisting of selected from the group consisting of Alzheimer's disease (AD), frontotemporal dementia, cerebral amyloid angiopathy (CAA), dementia with Lewy bodies (DLB), tauopathy, cerebrovascular disease, multiple sclerosis, vascular dementia, and traumatic brain injury and/or hemorrhage, said method comprising:
administering, or causing to be administered, to said mammal an effective amount of a synthetic exosome according to any one of claims 1-82, and/or a pharmaceutical formulation according to any one of claims 83-89.

96. The method according to any one of claims 92-95, wherein said pathology is Alzheimer's disease.

97. The method of claim 96, wherein said mammal is a mammal at risk for Alzheimer's disease.

98. The method of claim 97, wherein said mammal has a family history of Alzheimer's disease.

99. The method of claim 98, wherein said mammal has a genetic risk factor for Alzheimer's disease.

100. The method of claim 99, wherein the mammal has a familial Alzheimer's disease (FAD) mutation.

101. The method of claim 99, wherein said mammal has one copy of the ApoE4 allele.

102. The method of claim 99, wherein said mammal has two copies of the ApoE4 allele.

103. The method according to any one of claims 92-102, wherein said mammal is a human.

104. The method according to any one of claims 92-103, wherein said administering or causing to be administered comprises administering said synthetic exosome or said pharmaceutical formulation to said mammal.

105. The method according to any one of claims 92-103, wherein said administering or causing to be administered comprises prescribing or providing synthetic exosome or said pharmaceutical formulation to said mammal.
